(19) United States
(12) Patent Application Publication
(10) Pub. No.: US 2026/0198762 A1
(43) Pub. Date: Jul. 16, 2026

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Rito MURASE, Tokyo (JP)

(73) Assignee: FUJIFILM Corporation, Tokyo (JP)

(21) Appl. No.: 19/439,468

(22) Filed: Jan. 5, 2026

(30) Foreign Application Priority Data
Jan. 10, 2025 (JP) 2025-004389

(51) Int. Cl. A61B 1/005; A61B 1/00

(52) U.S. Cl. CPC A61B 1/0051; A61B 1/00006; A61B 1/000094; A61B 1/000096

(57) ABSTRACT

A medical support device includes a processor. The processor is configured to: input a plurality of endoscopic images obtained by imaging an inside of a luminal organ, by using an endoscope which is inserted into the luminal organ, to a trained model to cause the trained model to generate rotation information of the insertion part of the endoscope around a major axis; and generate shape information representing a shape of the insertion part based on the rotation information.

### CROSS-REFERENCE TO RELATED APPLICATIONS

This application claims priority under 35 USC 119 from Japanese Patent Application No. 2025-004389 filed on January 10, 2025, the disclosure of which is incorporated by reference herein.

### BACKGROUND

### 1. Technical Field

The present disclosure relates to a medical support device, an endoscope system, a medical support method, and a program.

### 2. Related Art

JP1999-019027A (JP-H11-019027A) discloses an endoscope shape detection device comprising a posture detection sensor unit in which a rotation angle detection unit that is provided in a plurality of points of an insertion part of an endoscope and detects a rotation angle of the provided point and converts the rotation angle into an electric signal is disposed on three orthogonal axes, a posture detection unit that samples an output of the posture detection sensor unit at predetermined intervals, and a shape detection unit that detects an insertion shape of the endoscope from a plurality of pieces of posture information sampled by a plurality of the posture detection units.

### SUMMARY

One embodiment according to the present disclosure provides a medical support device, an endoscope system, a medical support method, and a program that can estimate a shape of an insertion part of an endoscope in a case where the insertion part of the endoscope is inserted into a luminal organ without using an external device that recognizes the shape of the insertion part in the case where the insertion part of the endoscope is inserted into the luminal organ.

A first aspect according to the present disclosure is a medical support device comprising a processor, in which the processor is configured to: input a plurality of endoscopic images obtained by imaging an inside of a luminal organ, by using an endoscope which is inserted into the luminal organ, to a trained model to cause the trained model to generate rotation information of an insertion part of the endoscope around a major axis; and generate shape information representing a shape of the insertion part based on the rotation information.

A second aspect according to the present disclosure is the medical support device according to the first aspect, in which the rotation information is obtained based on an accumulated result in which changes in feature information obtained from the endoscopic image between the plurality of endoscopic images are accumulated.

A third aspect according to the present disclosure is the medical support device according to the second aspect, in which the feature information includes lumen position information indicating a position of a lumen included in the luminal organ in the endoscopic image, and the rotation information is obtained based on an accumulated result in which changes in the lumen position information between the plurality of endoscopic images are accumulated.

A fourth aspect according to the present disclosure is the medical support device according to the second or third aspect, in which the feature information includes gravity direction information for specifying a gravity direction, and the rotation information is obtained based on an accumulated result in which changes in the gravity direction information between the plurality of endoscopic images are accumulated.

A fifth aspect according to the present disclosure is the medical support device according to any one of the first to fourth aspects, in which the rotation information includes information on a rotation angle and a rotation direction of a second position, which are relative with respect to a first position of the insertion part, or information on a rotation angle and a rotation direction of the first position and the second position, which are absolute with respect to a reference angle.

A sixth aspect according to the present disclosure is the medical support device according to any one of the first to fifth aspects, in which the shape information includes information indicating that the insertion part forms a loop in a case where a distal end position of the insertion part is rotated by 180 degrees or more with respect to a base end position of the insertion part.

A seventh aspect according to the present disclosure is the medical support device according to the sixth aspect, in which the rotation information includes rotation direction information for specifying a rotation direction around the major axis, and the shape information includes loop classification information that classifies a shape of the loop based on the rotation direction information.

An eighth aspect according to the present disclosure is the medical support device according to the seventh aspect, in which the loop classification information includes information that classifies the loop into an α loop or an inverse α loop based on the rotation direction information.

A ninth aspect according to the present disclosure is the medical support device according to any one of the sixth to eighth aspects, in which the processor is configured to output information on a method of releasing the loop based on the rotation information and/or the shape information.

A tenth aspect according to the present disclosure is the medical support device according to any one of the first to ninth aspects, in which the processor is configured to input the plurality of endoscopic images and a plurality of images including a hand-held part of an operator in the insertion part to the trained model to cause the trained model to generate information based on information on rotation of the hand-held part around the major axis as the rotation information.

An eleventh aspect according to the present disclosure is the medical support device according to any one of the first to tenth aspects, in which the luminal organ is a large intestine.

A twelfth aspect according to the present disclosure is a medical support device comprising a processor, in which the processor is configured to input a plurality of endoscopic images obtained by imaging an inside of a luminal organ, by using an endoscope which is inserted into the luminal organ, to a trained model to cause the trained model to generate shape information representing a shape of an insertion part of the endoscope.

A thirteenth aspect according to the present disclosure is a medical support device comprising a processor, in which the processor is configured to input an accumulated result in which changes in feature information between a plurality of endoscopic images are accumulated, the feature information being included in each of the plurality of endoscopic images obtained by imaging an inside of a luminal organ, by using an endoscope which is inserted into the luminal organ, to a trained model to cause the trained model to generate shape information representing a shape of an insertion part of the endoscope.

A fourteenth aspect according to the present disclosure is an endoscope system comprising: the medical support device according to any one of the first to thirteenth aspects; and an output device that outputs the shape information generated by the medical support device and/or information based on the shape information generated by the medical support device.

A fifteenth aspect according to the present disclosure is a medical support method comprising: inputting a plurality of endoscopic images obtained by imaging an inside of a luminal organ, by using an endoscope which is inserted into the luminal organ, to a trained model to cause the trained model to generate rotation information of an insertion part of the endoscope around a major axis; and generating shape information representing a shape of the insertion part based on the rotation information.

A sixteenth aspect according to the present disclosure is a program causing a computer to execute a process comprising: inputting a plurality of endoscopic images obtained by imaging an inside of a luminal organ, by using an endoscope which is inserted into the luminal organ, to a trained model to cause the trained model to generate rotation information of an insertion part of the endoscope around a major axis; and generating shape information representing a shape of the insertion part based on the rotation information.

### BRIEF DESCRIPTION OF THE DRAWINGS

Exemplary embodiments of the technology of the disclosure will be described in detail based on the following figures, wherein:

FIG. 1 is a conceptual diagram showing an aspect example in which an endoscope system is used by a doctor;

FIG. 2 is a conceptual diagram showing an example of an overall configuration of the endoscope system;

FIG. 3 is a block diagram showing an example of a hardware configuration of an electrical system of the endoscope system;

FIG. 4 is a conceptual diagram showing an aspect example in which an insertion part of an endoscope forms a loop in a large intestine;

FIG. 5 is a conceptual diagram showing an example of features of an α loop and an inverse α loop;

FIG. 6 is a block diagram showing an example of main functions of a processor provided in a medical support device and an example of information stored in a storage;

FIG. 7 is a block diagram showing an example of the hardware configuration of the electrical system of the information processing device;

FIG. 8 is a conceptual diagram showing an example of an aspect in which training data is generated by the information processing device;

FIG. 9 is a conceptual diagram showing an example of an example image;

FIG. 10 is a conceptual diagram showing an example of training data generated in a case where a lumen is shown in one of a plurality of divided regions obtained by radially dividing the example image shown in FIG. 9;

FIG. 11 is a conceptual diagram showing an example of processing contents in the information processing device in a case in which the lumen recognition model is generated by training a model through machine learning using the training data;

FIG. 12 is a block diagram showing an example of the hardware configuration of the electrical system of the information processing device;

FIG. 13 is a conceptual diagram showing an example of a processing content in the information processing device in a case where a rotation recognition model is constructed based on a dataset group;

FIG. 14 is a conceptual diagram showing an example of processing contents in lumen recognition processing performed by a recognition unit;

FIG. 15 is a conceptual diagram showing an example of processing contents in rotation recognition processing performed by a recognition unit;

FIG. 16 is a conceptual diagram showing an example of processing contents of a control unit;

FIG. 17 is a flowchart showing an example of a flow of medical support processing;

FIG. 18 is a block diagram showing an example of functions of main units of a processor included in a medical support device and an example of information stored in a storage according to a second embodiment;

FIG. 19 is a conceptual diagram showing an example of an aspect in which training data according to the second embodiment is generated;

FIG. 20 is a conceptual diagram showing an example of processing contents in the information processing device in a case in which the gravity direction recognition model is generated by training a model through machine learning using the training data;

FIG. 21 is a conceptual diagram showing an example of a processing content in the information processing device in a case where a rotation recognition model according to the second embodiment is generated;

FIG. 22 is a conceptual diagram showing an example of a processing content of gravity direction recognition processing performed by a processor of the medical support device;

FIG. 23 is a conceptual diagram showing an example of a processing content of rotation recognition processing according to the second embodiment performed by a processor of the medical support device;

FIG. 24 is a conceptual diagram showing an example of a processing content in the information processing device in a case where a rotation recognition model according to a third embodiment is generated;

FIG. 25 is a conceptual diagram showing an example of a processing content of rotation recognition processing according to the third embodiment performed by a processor of the medical support device;

FIG. 26 is a conceptual diagram showing an example of a processing content in the information processing device in a case where a shape recognition model according to a fourth embodiment is generated;

FIG. 27 is a conceptual diagram showing an example of a processing content of shape recognition processing according to the fourth embodiment performed by a processor of the medical support device;

FIG. 28 is a conceptual diagram showing an example of a processing content in a case where release method information corresponding to shape information is derived by using a release information derivation table;

FIG. 29 is a conceptual diagram showing an example of a processing content in the information processing device in a case where a shape recognition model according to a fifth embodiment is generated;

FIG. 30 is a conceptual diagram showing an example of a processing content of shape recognition processing according to the fifth embodiment performed by a processor of the medical support device; and

FIG. 31 is a conceptual diagram showing an example of a series of processing in which a processor included in a computer issues a processing execution request to an external device via a network, the external device executes processing in response to the processing execution request, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings. It should be noted that the present disclosure can also be applied to a program and a computer program product.

First, terms used in the following description will be described.

CPU is an abbreviation for “central processing unit”. GPU is an abbreviation for “graphics processing unit”. GPGPU is an abbreviation for “general-purpose computing on graphics processing units”. NPU refers to an abbreviation for “Neural Processing Unit”. APU is an abbreviation for “accelerated processing unit”. TPU is an abbreviation for “tensor processing unit”. RAM is an abbreviation for "random-access memory". ASIC is an abbreviation for "application-specific integrated circuit". PLD is an abbreviation for “programmable logic device”. FPGA is an abbreviation for “field-programmable gate array”. SoC is an abbreviation for “system-on-a-chip”. SSD is an abbreviation for “solid state drive”. CD-ROM refers to an abbreviation for “Compact Disc Read Only Memory”. DVD-ROM refers to an abbreviation for “Digital Versatile Disc Read Only Memory”. USB is an abbreviation for "Universal Serial Bus". EL is an abbreviation for "electro-luminescence". CMOS is an abbreviation for "complementary metal-oxide-semiconductor". CCD is an abbreviation for "charge-coupled device". AI is an abbreviation for "artificial intelligence". WLI is an abbreviation for "white light imaging". BLI is an abbreviation for "blue light imaging". LCI is an abbreviation for "linked color imaging". NBI is an abbreviation for "narrow band imaging". I/F is an abbreviation for "interface". LAN is an abbreviation for "local area network". WAN is an abbreviation for "wide area network". 5G is an abbreviation for “5th generation mobile communication system”.

In the following description, a processor with a reference numeral (hereinafter, simply referred to as a “processor”) may be one computing device or a combination of a plurality of computing devices. Furthermore, the processor may be one type of computing device or may be a combination of a plurality of types of computing devices. Examples of the computing device include a CPU, a GPU, a GPGPU, an NPU, an APU, or a TPU.

In the following description, a memory with a reference numeral is a memory such as a RAM that temporarily stores information, and is used as a work memory by the processor.

In the following description, a storage with a reference numeral is one or a plurality of non-volatile storage devices that store various programs, various parameters, and the like. Examples of the non-volatile storage device include a flash memory, a magnetic disk, and a magnetic tape. Examples of the storage also include a cloud storage.

In the following embodiment, an external I/F with a reference numeral controls transmission and reception of various types of information between a plurality of devices connected to each other. An example of the external I/F is a USB interface. A communication I/F including a communication processor, an antenna, and the like may be applied to the external I/F. The communication I/F controls communication between a plurality of computers. Examples of a communication standard applied to the communication I/F include a wireless communication standard including 5G, Wi-Fi (registered trademark), and Bluetooth (registered trademark).

In the following embodiment, “A and/or B” is synonymous with “at least one of A or B”. That is, "A and/or B" may mean only A, may mean only B, or may mean a combination of A and B. In addition, in the present specification, in a case in which three or more matters are expressed with the connection of “and/or”, the same concept as “A and/or B” is applied.

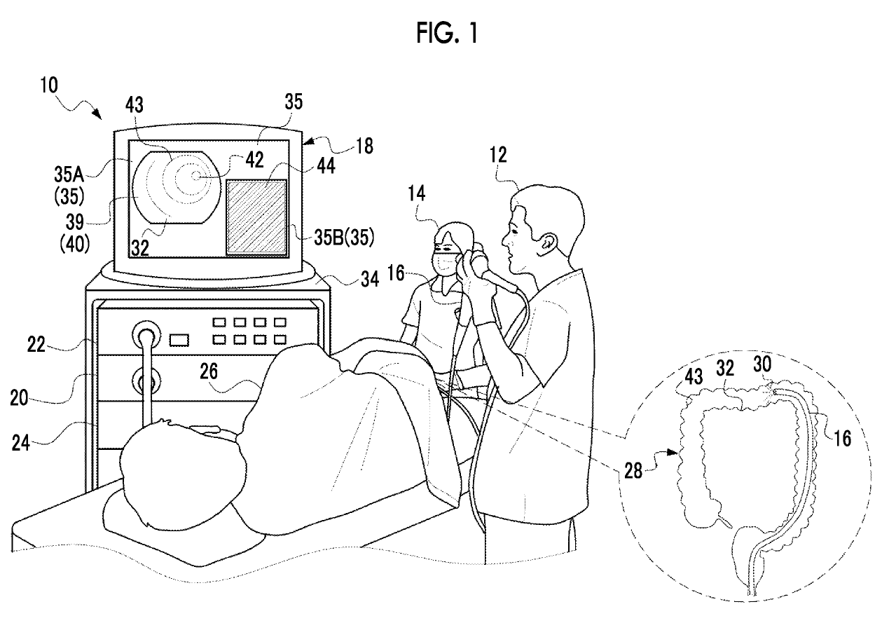

First Embodiment FIG. 1 is a conceptual diagram showing an example of an aspect in which an endoscope system 10 is used. As shown in FIG. 1, an endoscope system 10 is used by a doctor 12 in an endoscopy and the like. A staff member 14, such as a nurse, assists with the endoscop.

The endoscope system 10 is connected to a communication device (not shown) in a communicable manner, and information obtained by the endoscope system 10 is transmitted to the communication device. Examples of the communication device include a server, a personal computer, and/or a tablet terminal that manage various types of information, such as electronic medical records. The communication device receives the information transmitted from the endoscope system 10 and executes processing using the received information (for example, processing of storing the information in the electronic medical record or the like).

The endoscope system 10 comprises an endoscope 16, a display device 18, a light source device 20, a control device 22, and a medical support device 24. In the first embodiment, the endoscope system 10 is an example of an “endoscope system” according to the present disclosure, the endoscope 16 is an example of an “endoscope” according to the present disclosure, the display device 18 is an example of an “output device” according to the present disclosure, and the medical support device 24 is an example of a “medical support device” according to the present disclosure.

The endoscope system 10 is a modality for performing a medical examination on a large intestine 28, which is a luminal organ included in a body of a subject 26 (for example, a patient), by using the endoscope 16. In the first embodiment, the large intestine 28 is a target to be observed by the doctor 12.

The endoscope 16 is used by the doctor 12 and is inserted into the body of the subject 26. In the first embodiment, the endoscope 16 is inserted into the large intestine 28 of the subject 26. The large intestine 28 in the present embodiment is an example of a "luminal organ" according to the first present disclosure.

The endoscope system 10 images an inside of the large intestine 28 including a lumen 42 by using the endoscope 16 inserted into the large intestine 28 of the subject 26, and performs various medical treatments on the large intestine 28 as necessary.

The large intestine 28 has the lumen 42. The endoscope 16 is inserted into the lumen 42. A position of the lumen 42 in the large intestine 28 can be medically specified based on a form pattern of a plurality of folds 43 (for example, a shape, an orientation, and the like of the plurality of folds 43) which are characteristic regions in the large intestine 28. In the first present embodiment, although the details will be described later, the position of the lumen 42 is recognized by AI that has been trained using various types of information, such as the form pattern of the plurality of folds 43, through machine learning, and a result of the recognition is provided as visually ascertainable information to the doctor 12. The lumen 42 in the present embodiment is an example of a "lumen" according to the first present disclosure.

The endoscope system 10 acquires an image showing an aspect including the lumen 42 in the large intestine 28 by imaging the inside of the large intestine 28 including the lumen 42, and outputs the acquired image. In the first present embodiment, the endoscope system 10 has an optical imaging function of emitting light 30 in the large intestine 28 and imaging reflected light obtained by being reflected by an intestinal wall 32 of the large intestine 28.

In addition, here, the endoscopy of the large intestine 28 is given as an example. However, this is only an example, and the present disclosure is applicable to the endoscopy of a luminal organ such as an esophagus, a stomach, a duodenum, or a trachea.

The light source device 20, the control device 22, and the medical support device 24 are installed in a wagon 34. The wagon 34 is provided with a plurality of tables along an up-down direction, and the medical support device 24, the light source device 20, and the control device 22 are installed from a lower table to an upper table. Furthermore, the display device 18 is installed on an uppermost table in the wagon 34.

The control device 22 controls the entire endoscope system 10. The control device 22 performs various types of processing on an image obtained by imaging the wall of the large intestine 32 by the endoscope 16. In addition, the medical support device 24 executes AI-based processing or the like on the image that has been subjected to various types of processing by the control device 22, under the control of the control device 22, and outputs various types of information including a processing result of the AI-based processing or the like. Examples of an output destination of the various types of information include the display device 18, a stationary storage medium (for example, a storage mounted in the endoscope system 10, a storage of a server or the like that is connected to the endoscope system 10 in a communicable manner, and the like), and/or a portable storage medium (for example, a memory card, a USB flash drive, and the like).

The display device 18 displays various types of information (for example, various types of information output from the medical support device 24). Examples of the display device 18 include a liquid crystal display and an EL display. In addition, a tablet terminal with a display may be used instead of the display device 18 or together with the display device 18.

A screen 35 is displayed on the display device 18. A plurality of display regions are included in the screen 35. The plurality of display regions are arranged in the screen 35. In the example shown in FIG. 1, a first display region 35A and a second display region 35B are shown as examples of the plurality of display regions. The first display region 35A has a larger size than the second display region 35B. The first display region 35A is used as a main display region, and the second display region 35B is used as a sub-display region. A size relationship between the first display region 35A and the second display region 35B is not limited to this and may be any size relationship that falls within the screen 35.

An endoscopic video image 39 is displayed in the first display region 35A. The endoscopic video image 39 is obtained by executing various types of processing on a plurality of images arranged in time series obtained by imaging the inside of the large intestine 28 of the subject 26 with the endoscope 16. The intestinal wall 32 shown in the endoscopic video image 39 includes the lumen 42 as a region of interest (that is, an observation target region) at which the doctor 12 gazes, and the doctor 12 can visually recognize the aspect of the intestinal wall 32 including the lumen 42 through the endoscopic video image 39.

The image displayed in the first display region 35A is one frame 40 included in a video image including a plurality of frames 40 arranged in time series. That is, the plurality of frames 40 arranged in time series are displayed in the first display region 35A at predetermined frame rates (for example, a dozen frames/second or a few dozen frames/second). In the first embodiment, the frame 40 is an example of an “endoscopic image” according to the present disclosure.

An example of the video image displayed in the first display region 35A is a video image in a live view mode. The live view mode is merely an example, and the video image may be a video image, such as a video image in a post view mode, that is temporarily stored in a memory or the like and then displayed. In addition, each frame included in a recording video image stored in the memory or the like may be reproduced and displayed as the endoscope video image 39 on the screen 35 (for example, in the first display region 35A).

The second display region 35B is displayed on the lower right side of the screen 35 in a front view. The second display region 35B may be displayed at any position as long as the position is within the screen 35 of the display device 18, but is preferably displayed at a position that is comparable with the endoscopic video image 39. Auxiliary information 44 for assistance of the doctor 12 in a medical determination or the like is displayed in the second display region 35B. The auxiliary information 44 is information to be referred to by the doctor 12. Examples of the auxiliary information 44 include various types of information on the subject 26 into which the endoscope 16 is inserted and/or various types of information obtained by executing a medical support process described later.

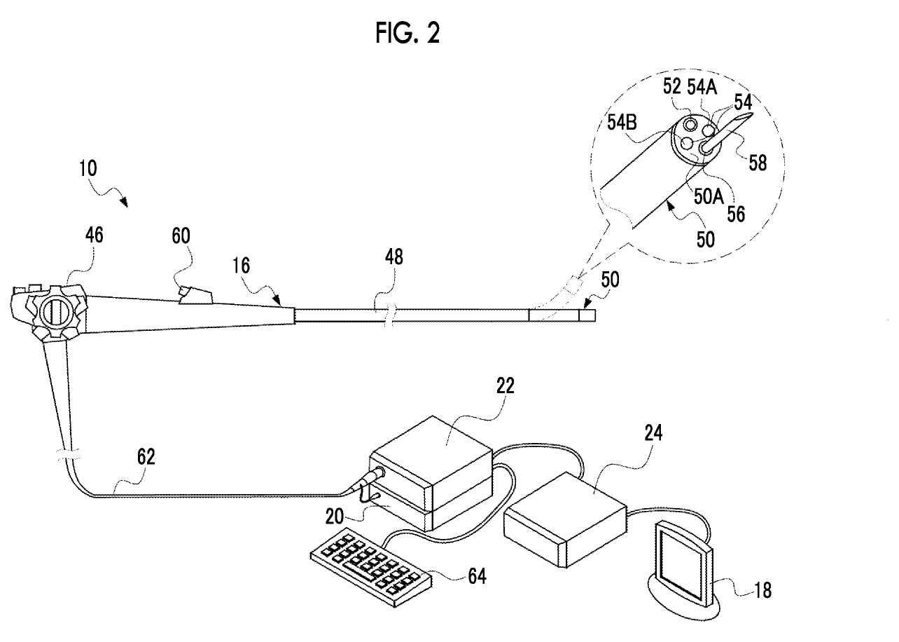

FIG. 2 is a conceptual diagram showing an example of an overall configuration of the endoscope system 10. As shown in FIG. 2, the endoscope 16 comprises an operating unit 46 and an insertion part 48. The insertion part 48 is formed in a tubular shape and is partially bent by operating the operating unit 46. The insertion part 48 is inserted into the large intestine 28 while being curved along the shape of the large intestine 28 (see FIG. 1) in accordance with the operation of the operating unit 46 performed by the doctor 12 (see FIG. 1). In the first embodiment, the insertion part 48 is an example of an “insertion part” according to the present disclosure.

A camera 52, an illumination device 54, and a treatment tool opening 56 are provided at a distal end portion 50 of the insertion part 48. A portion of the camera 52 (for example, an imaging optical system) and a portion of the illumination device 54 (for example, an irradiation optical system) are exposed from a distal end surface 50A of the distal end portion 50.

The camera 52 is mounted in the endoscope 16 and is inserted into a body cavity (here, for example, the lumen 42) of the subject 26 to image the observation target region. Examples of the camera 52 include a CMOS camera. However, this is merely an example, and other types of cameras, such as CCD cameras, may be used. In the first present embodiment, the camera 52 generates an image showing the aspect including the lumen 42 in the large intestine 28 by imaging the inside of the large intestine 28 including the lumen 42. The image generated by the camera 52 is an image of which an outer shape is circular. For example, the image generated by the camera 52 is processed into a shape in which an upper end portion and a lower end portion are masked, by the control device 22. Accordingly, as shown in FIG. 1, an image in which an upper end edge and a lower end edge are linear and a left side edge and a right side edge are arc-shaped is generated as the frame 40.

The illumination device 54 includes illumination windows 54A and 54B. The illumination windows 54A and 54B are provided on the distal end surface 50A. The illumination device 54 emits the light 30 (see FIG. 1) through the illumination windows 54A and 54B. Examples of the type of the light 30 emitted from the illumination device 54 include light for WLI (for example, white light), light for LCI (for example, light obtained by combining red light, green light, and blue light), light for BLI (for example, blue light), and/or light for NBI (for example, light obtained by combining blue light and green light). The camera 52 images the inside of the large intestine 28 using an optical method in a state in which the inside of the large intestine 28 is irradiated with the light 30 (see FIG. 1) by the illumination device 54.

The treatment tool opening 56 is an opening through which a treatment tool 58 protrudes from the distal end part 50. Furthermore, the treatment tool opening 56 is also used as a suction port for suctioning blood, internal contaminants, and the like and a sending-out port for sending out fluid. Examples of the fluid include a gas (for example, air or the like) and/or a liquid (for example, water or the like).

A treatment tool insertion opening 60 is formed in the operation unit 46, and the treatment tool 58 is inserted into the insertion part 48 through the treatment tool insertion opening 60. The treatment tool 58 passes through the insertion part 48 to protrude from the treatment tool opening 56 to the outside. In the example shown in FIG. 2, an aspect is shown in which a biopsy needle protrudes through the treatment tool opening 56 as the treatment tool 58. Here, the puncture needle is given as an example of the treatment tool 58. However, this is only an example. The treatment tool 58 may be grasping forceps, a papillotomy knife, a snare, a catheter, a guide wire, a cannula, and/or a puncture needle with a guide sheath.

The endoscope 16 is connected to the light source device 20 and the control device 22 via a universal cord 62. The medical support device 24 and a reception device 64 are connected to the control device 22. Furthermore, the display device 18 is connected to the medical support device 24. That is, the control device 22 is connected to the display device 18 through the medical support device 24.

In addition, here, the medical support device 24 is given as an example of an external device for expanding the functions of the control device 22. Therefore, a form in which the control device 22 and the display device 18 are indirectly connected to each other through the medical support device 24 is given as an example. However, this is only an example. For example, the display device 18 may be directly connected to the control device 22. In this case, for example, the functions of the medical support device 24 may be provided in the control device 22, or the control device 22 may be provided with a function of directing a server (not illustrated) to execute the same process as the process (for example, a medical support process which will be described below) performed by the medical support device 24, receiving a result of the process by the server, and using the result.

The receiving device 64 receives an instruction from the doctor 12 and outputs the received instruction as an electric signal to the control device 22. Examples of the receiving device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light 30 (see FIG. 1) to the illumination device 54. A light guide is provided in the illumination device 54, and the light 30 supplied from the light source device 20 is emitted from the illumination windows 54A and 54B through the light guide. The control device 22 causes the camera 52 to perform the imaging in a state in which the light 30 is emitted from the illumination windows 54A and 54B. The control device 22 generates the plurality of frames 40 arranged in time series by processing the outer shape of the image obtained by imaging performed by the camera 52 or adjusting an image quality or the like of the image. The control device 22 outputs the endoscopic video image 39 including the plurality of generated frames 40 arranged in time series to a predetermined output destination (for example, the medical support device 24).

The medical support device 24 executes various types of processing on the endoscopic video image 39 input from the control device 22 to support a medical treatment (here, for example, endoscopy). The medical support device 24 outputs the endoscopic video image 39, which has been subjected to various types of processing, to a predetermined output destination (for example, the display device 18).

Here, the form example has been described in which the endoscopic video image 39 output from the control device 22 is output to the display device 18 via the medical support device 24, but this is merely an example. For example, an aspect may be adopted in which the control device 22 and the display device 18 are connected to each other, and the endoscopic video image 39, which has been subjected to various types of processing by the medical support device 24, is displayed on the display device 18 via the control device 22.

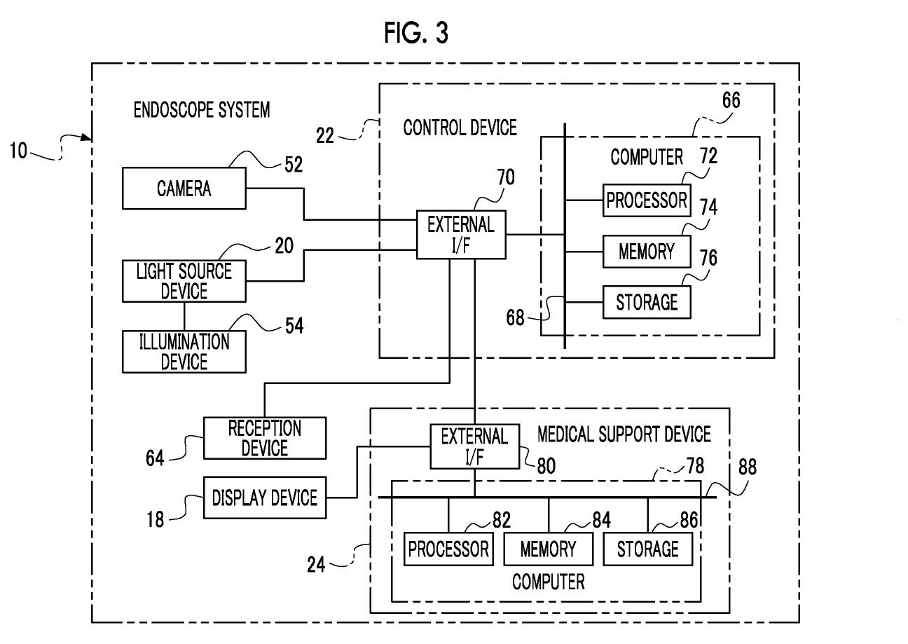

FIG. 3 is a block diagram showing an example of a hardware configuration of an electrical system of the endoscope system 10. As shown in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as "first external devices") outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 through the external I/F 70. In addition, the processor 72 acquires an image generated by imaging the inside of the large intestine 28 (see FIG. 1) with the camera 52 via the external I/F 70, and performs various types of processing on the acquired image to generate the endoscopic video image 39 (see FIG. 1).

The light source device 20 is connected to the external I/F 70 as one of the first external devices, and the external I/F 70 transmits and receives various types of information between the light source device 20 and the processor 72. The light source device 20 supplies the light 30 to the illumination device 54 under the control of the processor 72. The illumination device 54 emits the light 30 supplied from the light source device 20.

As one of the first external devices, the receiving device 64 is connected to the external I/F 70. The processor 72 acquires the instruction received by the receiving device 64 via the external I/F 70 and executes a process corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the first present embodiment, the computer 78 is an example of a "computer" according to the present disclosure, and the processor 82 is an example of a "processor" according to the present disclosure.

Since a hardware configuration (that is, the processor 82, the memory 84, and the storage 86) of the computer 78 is basically the same as the hardware configuration of the computer 66, a description of the hardware configuration of the computer 78 will be omitted here.

The external I/F 80 transmits and receives various types of information between one or more devices (hereinafter, also referred to as "second external devices") outside the medical support device 24 and the processor 82.

As one of the second external devices, the control device 22 is connected to the external I/F 80. In the example shown in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic video image 39 (see FIG. 1) from the processor 72 of the control device 22 via the external I/Fs 70 and 80, and executes various types of processing on the acquired endoscopic video image 39. The various types of processing performed by the processor 82 include AI-based processing (for example, lumen recognition processing 166 that is processing using a lumen recognition model 92 described below and rotation recognition processing 172 that is processing using a rotation recognition model 94).

The display device 18 is connected to the external I/F 80 as one of the second external devices. The processor 82 controls the display device 18 via the external I/F 80 such that various types of information (for example, the endoscopic video image 39 that has been subjected to various types of processing) are displayed on the display device 18.

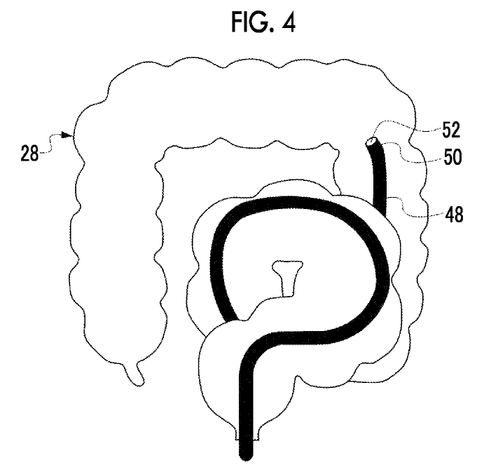

The large intestine 28 has a complicated shape, and it may be difficult to insert the insertion part 48. For example, as shown in FIG. 4, in the sigmoid colon and the transverse colon that are not fixed to the abdominal wall, the insertion part 48 may form a loop due to a shape of the intestinal tract, a pressure of the intestinal tract, a physical influence on the large intestine 28 caused by operating the insertion part 48, and the like. In a case where such a loop is formed, it is difficult to insert the insertion part 48 into the large intestine 28 on the inside. In a case where the insertion of the insertion part 48 is difficult, the subject 26 is also subjected to a physical burden. Therefore, it is very important to allow the doctor 12 to understand in real time what shape the insertion part 48 has in the large intestine 28.

As a device for allowing the doctor 12 to understand the shape of the insertion part 48 in the large intestine 28, an endoscope shape observation device in which a dedicated scope that generates a magnetic field and an external device are combined is known. The endoscope shape observation device can display the shape of the insertion part 48 on the display in real time, but since the dedicated scope and the external device are required, there is a problem that it takes time to prepare and operate the device and the convenience is insufficient.

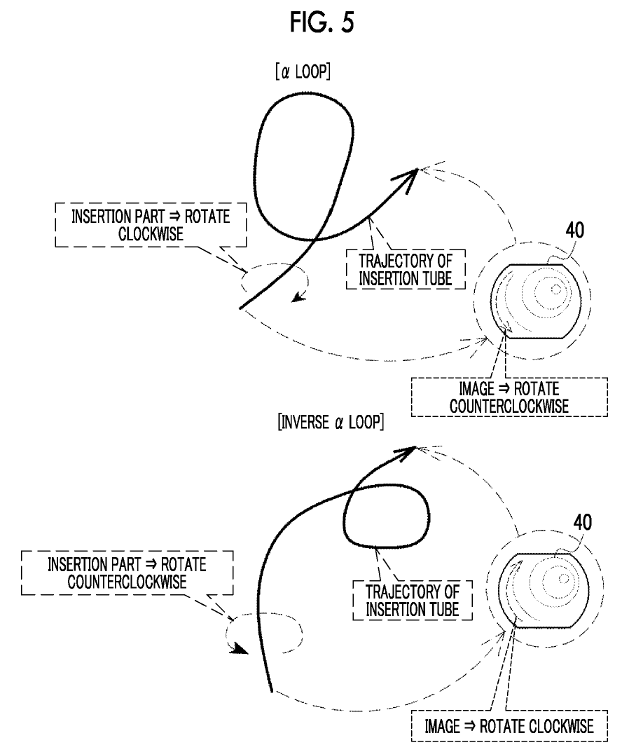

In addition, the method known in the related art also has a problem that it is difficult to specifically specify the loop shape of the insertion part 48 in the large intestine 28. Examples of a representative loop shape include an α loop and an inverse α loop. For example, as shown in FIG. 5, the α loop is a loop formed by the insertion part 48 rotating clockwise by 180 degrees or more around a major axis of the insertion part 48 (that is, around the major axis in a case of viewing the distal end side from the base end side of the insertion part 48 along the major axis of the insertion part 48) in the large intestine 28. On the other hand, the inverse α loop is a loop formed by the insertion part 48 rotating counterclockwise by 180 degrees or more around the major axis of the insertion part 48 in the large intestine 28. The α loop and the inverse α loop mainly occur in intestinal tract parts having high mobility, such as the sigmoid colon and the transverse colon.

As described above, in a case where the α loop is formed and in a case where the inverse α loop is formed, the insertion part 48 rotates around the major axis by 180 degrees or more, but the rotation direction of the insertion part 48 around the major axis is different. In a case where the α loop is formed, the plurality of frames 40 obtained in a process until the α loop is formed rotate counterclockwise by 180 degrees or more around the center of the frame 40. On the other hand, in a case where the inverse α loop is formed, the plurality of frames 40 obtained in a process until the inverse α loop is formed rotate clockwise by 180 degrees or more around the center of the frame 40.

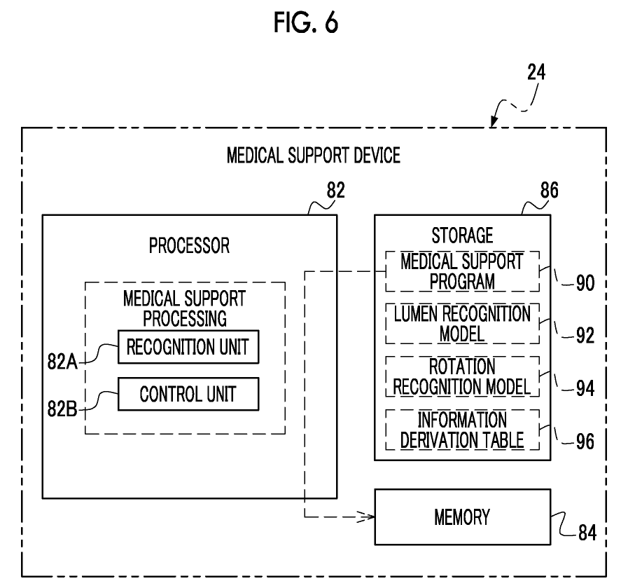

Therefore, in the first embodiment, in order to solve the above-described problem, as shown in FIG. 6 as an example, the processor 82 executes medical support processing by using the fact that different phenomena occur on the frame 40 in a case where the α loop is formed and in a case where the inverse α loop is formed.

FIG. 6 is a block diagram showing an example of main functions of the processor 82 included in the medical support device 24 and an example of the information stored in the storage 86. As shown in FIG. 6, a medical support program 90 is stored in the storage 86. The medical support program 90 in the first present embodiment is an example of a "program" according to the present disclosure.

The processor 82 reads out the medical support program 90 from the storage 86, and executes the readout medical support program 90 on the memory 84 to perform medical support processing. The medical support processing is implemented by the processor 82 operating as a recognition unit 82A and a control unit 82B in accordance with the medical support program 90 executed on the memory 84.

The storage 86 stores a lumen recognition model 92, a rotation recognition model 94, and a information derivation table 96. Although the details will be described later, each of the lumen recognition model 92 and the rotation recognition model 94 is a machine learning model and is used by the recognition unit 82A. Examples of the machine learning model include a neural network (for example, a recurrent neural network, a two-dimensional convolutional neural network, and/or a three-dimensional convolutional neural network). The information derivation table 96 is used by the control unit 82B. Examples of the information derivation table 96 include a look-up table represented as a table in which a pair of an input value and an output value corresponding to the input value is defined in advance.

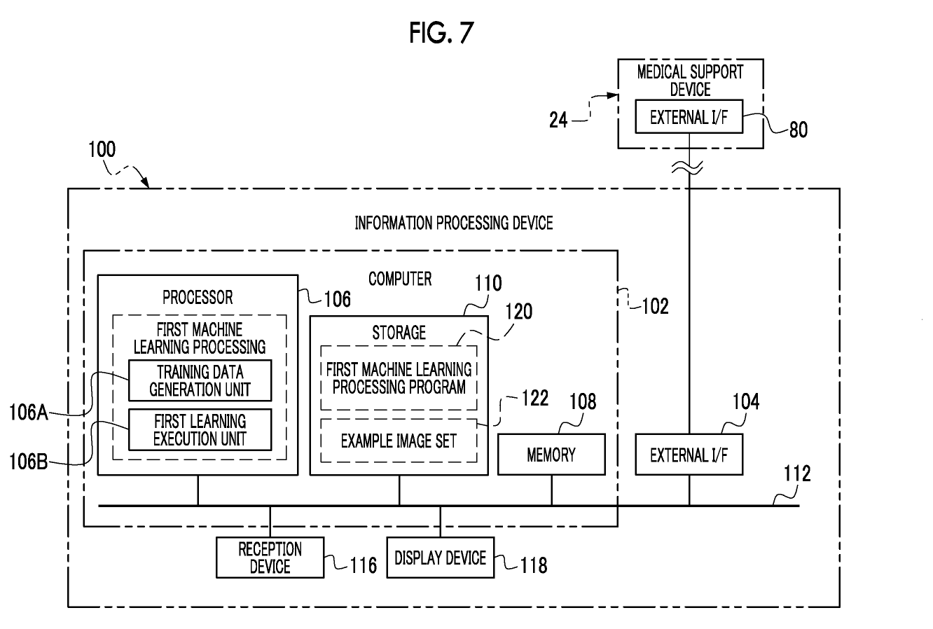

FIG. 7 is a block diagram showing an example of a hardware configuration of an electrical system of an information processing device 100 used to generate the lumen recognition model 92 and the rotation recognition model 94. As shown in FIG. 7, the information processing device 100 comprises a computer 102 and an external I/F 104. The computer 102 comprises a processor 106, a memory 108, and a storage 110. The processor 106, the memory 108, the storage 110, and the external I/F 104 are connected to a bus 112.

It should be noted that a hardware configuration (that is, the processor 106, the memory 108, and the storage 110) of the computer 102 is essentially the same as the hardware configuration of the computer 66, and thus the description of the hardware configuration of the computer 102 will be omitted here.

The information processing device 100 comprises a reception device 116. The reception device 116 is, for example, a keyboard and/or a mouse, and receives an instruction from a user of the information processing device 100 and the like. The reception device 116 is connected to the bus 112. The processor 106 acquires the instruction received by the reception device 116 and operates in accordance with the acquired instruction.

A display device 118 displays various types of information including the image. Examples of the display device 118 include a liquid crystal display and an EL display. The display device 118 is connected to the bus 112. The processor 106 displays the results obtained by executing various types of processing on the display device 118.

The external I/F 104 transmits and receives various types of information between one or more devices (hereinafter, also referred to as "third external devices") existing outside the information processing device 100 and the processor 106. The medical support device 24 is connected to the external I/F 104 as one of the third external devices. In the example shown in FIG. 7, the external I/F 80 of the medical support device 24 is connected to the external I/F 104. The external I/F 104 controls the transmission and reception of various types of information between the processor 82 (see FIGS. 3 and 6) of the medical support device 24 and the processor 106 of the information processing device 100. For example, the information processing device 100 generates the lumen recognition model 92 and the rotation recognition model 94, and transmits the generated lumen recognition model 92 and rotation recognition model 94 to the medical support device 24 via the external I/Fs 80 and 104 in response to a request from the medical support device 24.

A first machine learning processing program 120 is stored in the storage 110. The processor 106 reads out the first machine learning processing program 120 from the storage 110, and executes the readout first machine learning processing program 120 on the memory 108 to perform first machine learning processing. The first machine learning processing is implemented by the processor 106 operating as a training data generation unit 106A and a first learning execution unit 106B in accordance with the first machine learning processing program 120 executed on the memory 108.

An example image set 122 is stored in the storage 110. Although the details will be described later, the example image set 122 is used by the training data generation unit 106A.

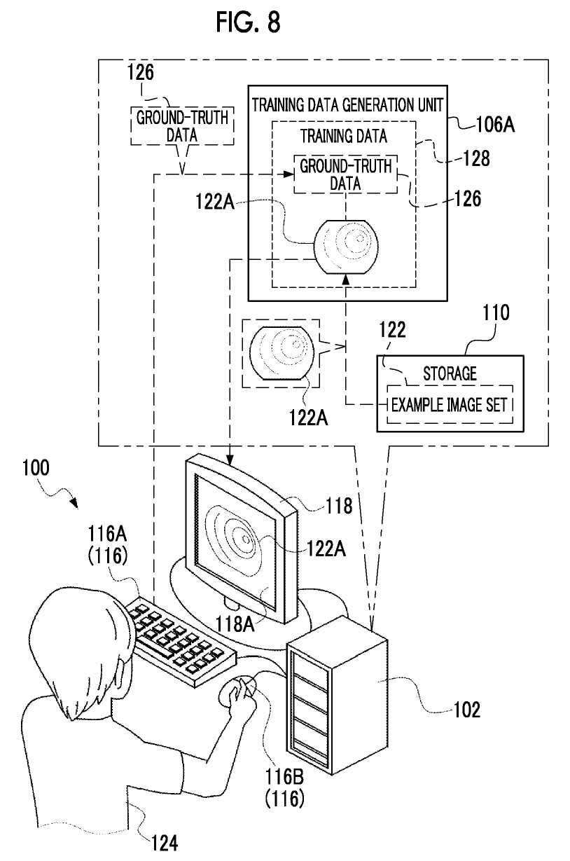

FIG. 8 is a conceptual diagram showing an example of processing contents in the training data generation unit 106A. As shown in FIG. 8, the information processing device 100 is used by an annotator 124. The annotator 124 means an operator who adds annotations for machine learning to given data (that is, an operator who performs labeling).

In the example shown in FIG. 8, a keyboard 116A and a mouse 116B are shown as examples of the reception device 116. The annotator 124 issues an instruction to the computer 102 via the keyboard 116A and the mouse 116B.

The example image set 122 includes a plurality of example images 122A showing different contents. The example image 122A is an image determined in advance as a medical image to be used for object recognition processing (for example, processing in which the recognition unit 82A recognizes the lumen 42 based on the frame 40 and the lumen recognition model 92). The image determined in advance as the medical image to be used for the object recognition processing is an image corresponding to the frame 40. In other words, the image corresponding to the frame 40 can also be referred to as an image that represents the frame 40. In other words, the image that represents the frame 40 can also be referred to as an image showing a sample of the frame 40. Here, a first example of the image showing the sample of the frame 40 is an image obtained by actually imaging the inside of the large intestine with the camera. A second example of the image showing the sample of the frame 40 is a virtually created image (for example, an image generated by generative AI, such as Stable Diffusion or Midjourney).

The training data generation unit 106A acquires the example image 122A from the example image set 122 in response to the instruction received by the reception device 116. The training data generation unit 106A displays the example image 122A on a screen 118A of the display device 118. In a state in which the example image 122A is displayed on the screen 118A, the annotator 124 indicates a lumen correspondence position, which is the position of the lumen shown in the example image 122A in the example image 122A, with respect to the training data generation unit 106A via the reception device 116. The training data generation unit 106A associates ground truth data 126 with the example image 122A based on the lumen correspondence position indicated via the reception device 116, to generate training data 128. The association of the ground truth data 126 with the example image 122A is implemented by adding an annotation capable of specifying the lumen correspondence position as the ground truth data 126 to the lumen correspondence position in the example image 122A.

In this way, the training data generation unit 106A repeatedly executes the processing of associating the ground truth data 126 with each of the example images 122A included in the example image set 122 in response to the instruction issued from the annotator 124, to generate a plurality of pieces of training data 128.

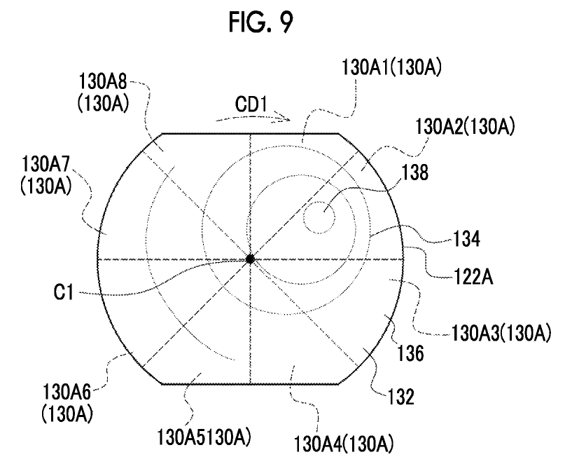

FIG. 9 is a conceptual diagram showing an example of a composition of the example image 122A. As shown in FIG. 9, a large intestine 132 is shown in the example image 122A. In the example shown in FIG. 9, an intestinal wall 136 in which the plurality of folds 134 are formed and a lumen 138 are shown in the example image 122A.

The example image 122A is divided into a plurality of divided regions 130A. Eight divided regions 130A1 to 130A8 are included in the plurality of divided regions 130A. The divided regions 130A1 to 130A8 are regions that radially exist from a center C1 of the example image 122A toward an outer edge of the example image 122A, and are disposed along a circumferential direction CD1 (in other words, around the center C1) of the example image 122A.

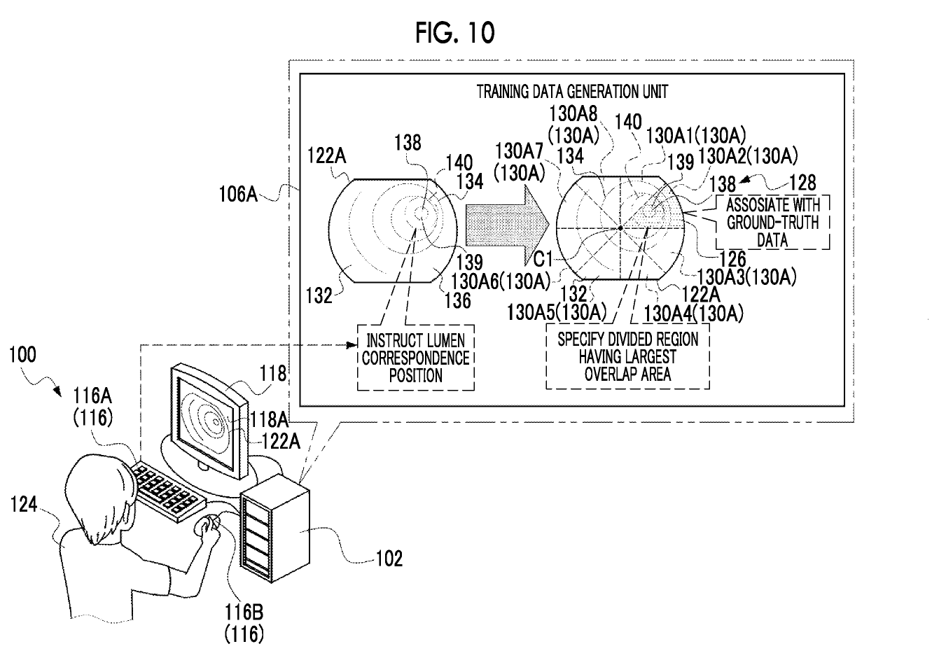

FIG. 10 is a conceptual diagram illustrating an example of a method in which the training data generation unit 106A associates the ground-truth data 126 with the example image 122A to generate the training data 128.

As shown in FIG. 10, in a state in which the example image 122A is displayed on the screen 118A, the annotator 124 indicates the lumen correspondence position 139, which is the position of the lumen 138 shown in the example image 122A in the example image 122A, with respect to the training data generation unit 106A via the reception device 116. The training data generation unit 106A displays a circular frame 140 in a superimposed manner on the example image 122A in response to the instruction received by the reception device 116, and disposes the frame 140 at a position surrounding the lumen 138 shown in the example image 122A. The frame 140 is a mark that defines the lumen correspondence position 139 in the example image 122A. That is, a position of a region surrounded by the frame 140 in the example image 122A is the lumen correspondence position 139. The size and the position of the frame 140 are freely changed on the screen 118A in response to the instruction received by the reception device 116. Here, the shape of the frame 140 is a circular shape, but the shape may be other than the circular shape. The size of the frame 140 can be changed in response to the instruction received by the reception device 116.

The annotator 124 issues a confirmation instruction, which is an instruction to confirm the lumen correspondence position 139, to the training data generation unit 106A via the reception device 116 in a state in which the frame 140 is disposed at the position surrounding the lumen 138. As a result, the training data generation unit 106A confirms the lumen correspondence position 139.

The training data generation unit 106A specifies the divided region 130A having a largest overlap area with the frame 140 that defines the lumen correspondence position 139 among the plurality of divided regions 130A. Then, the training data generation unit 106A generates the training data 128 by associating the ground truth data 126 with the specified divided region 130A (in the example shown in FIG. 10, the divided region 130A2) as the annotation capable of specifying the divided region 130A in which the lumen 138 is shown.

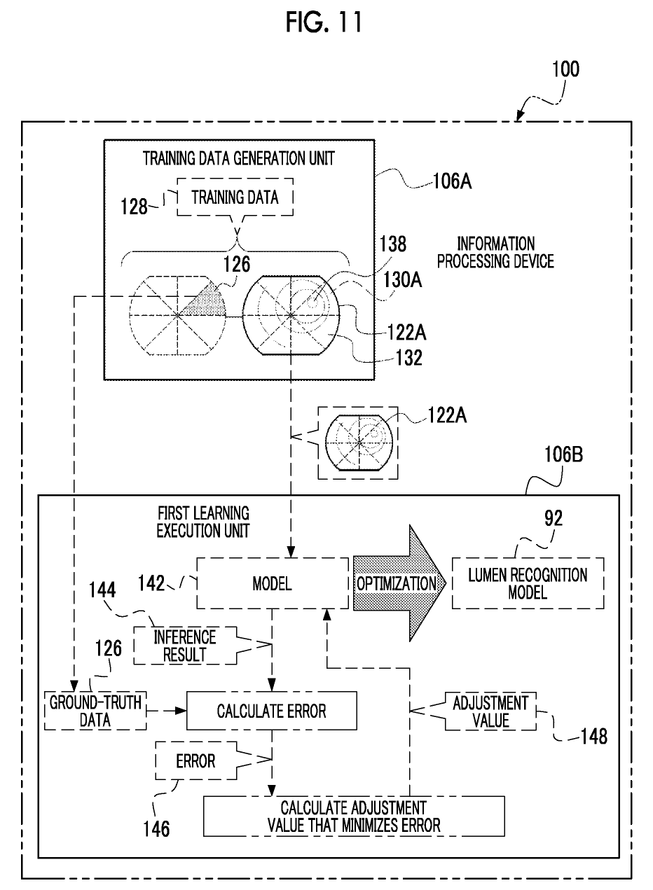

FIG. 11 is a conceptual diagram showing an example of an aspect in which the first learning execution unit 106B executes machine learning using the training data 128 to generate the lumen recognition model 92.

As shown in FIG. 11, in the information processing device 100, the first learning execution unit 106B acquires the training data 128 generated by the training data generation unit 106A. The first learning execution unit 106B executes the machine learning using the training data 128. Hereinafter, the details will be described with reference to FIG. 11.

In the example shown in FIG. 11, the first learning execution unit 106B executes processing using a model 142. Examples of the model 142 include a neural network. Examples of the neural network include a recurrent neural network, a two-dimensional convolutional neural network, and/or a three-dimensional convolutional neural network. The first learning execution unit 106B inputs the example image 122A included in the training data 128 to the model 142. In a case in which the example image 122A is input, the model 142 performs an inference to output an inference result 144. The first learning execution unit 106B calculates an error 146 between the inference result 144 and the ground truth data 126 included in the training data 128.

The first learning execution unit 106B calculates a plurality of adjustment values 148 for minimizing the error 146. Then, the first learning execution unit 106B adjusts a plurality of optimization variables in the model 142 by using the plurality of adjustment values 148, to optimize the model 142. Examples of the plurality of optimization variables in the model 142 include a weight indicating a strength of a connection between layers (in other words, a strength of a connection between neurons), and a bias that is a value for controlling activation of a neuron (in other words, a value used to adjust an output of the neuron).

The first learning execution unit 106B repeatedly executes learning processing of inputting the example image 122A to the model 142, calculating the error 146, calculating the plurality of adjustment values 148, and adjusting the plurality of optimization variables in the model 142 using the plurality of pieces of training data 128. That is, the first learning execution unit 106B adjusts the plurality of optimization variables in the model 142 using the plurality of adjustment values 148 calculated such that the error 146 is minimized for each of the plurality of example images 122A included in the plurality of pieces of training data 128, to optimize the model 142. The lumen recognition model 92 is generated by optimizing the model 142 in this manner. The lumen recognition model 92 is transmitted from the information processing device 100 to the medical support device 24 via the external I/Fs 80 and 104 (see FIG. 7), and is received by the medical support device 24. Then, in the medical support device 24, the lumen recognition model 92 is stored in the storage 86 by the processor 82 (see FIG. 6). The lumen recognition model 92 stored in the storage 86 is used by the recognition unit 82A (see FIG. 6).

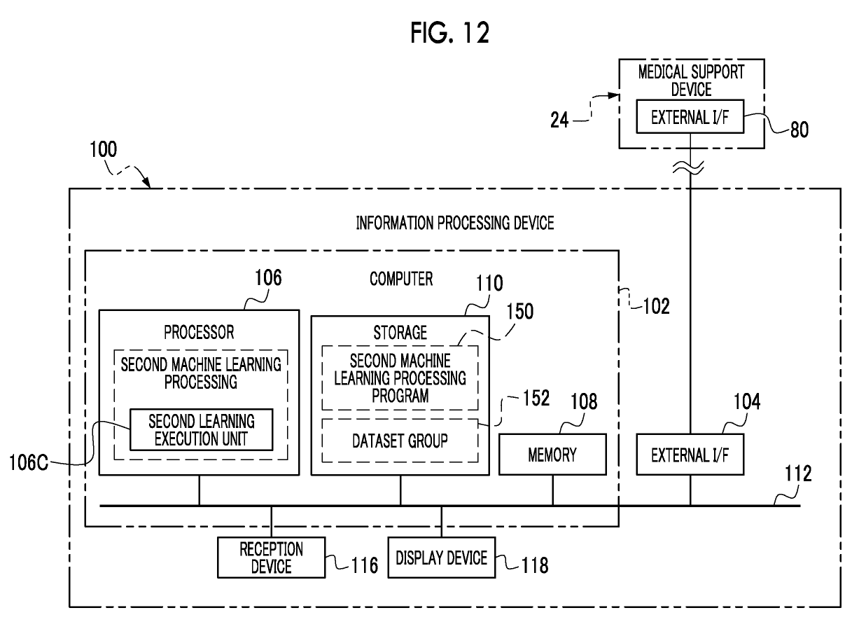

As shown in FIG. 12 as an example, in the information processing device 100, a second machine learning processing program 150 is stored in the storage 110. The processor 106 reads out the second machine learning processing program 150 from the storage 110, and executes the readout second machine learning processing program 150 on the memory 108 to perform a second machine learning processing. The second machine learning processing is implemented by the processor 106 operating as a second learning execution unit 106C in accordance with the second machine learning processing program 150 executed on the memory 108.

In addition, a dataset group 152 is stored in the storage 110. The dataset group 152 is used by the second learning execution unit 106C.

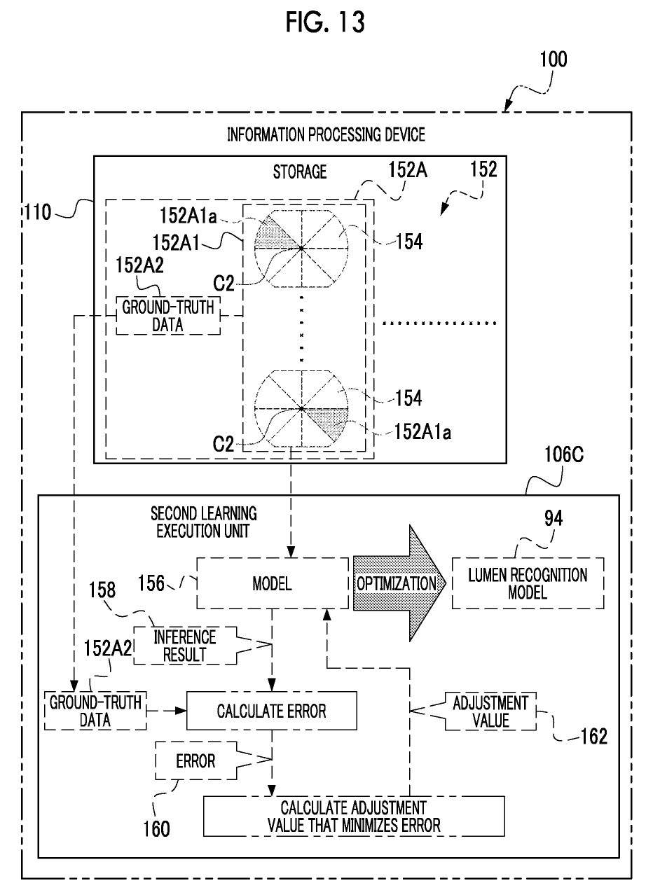

FIG. 13 is a conceptual diagram showing an example of an aspect in which the rotation recognition model 94 is generated by performing machine learning using the dataset group 152 by the second learning execution unit 106C.

As shown in FIG. 13, the dataset group 152 is a set of a plurality of datasets 152A. The plurality of datasets 152A have different contents. The dataset 152A is training data in which a lumen position information set 152A1 and ground-truth data 152A2 are associated with each other. The dataset 152A is generated by associating the ground-truth data 152A2 with the lumen position information set 152A1 in the same manner as the training data 128 shown in FIG. 8 is generated by the training data generation unit 106A.

The ground-truth data 152A2 is data that can specify a rotation direction and a rotation angle of an insertion part of an endoscope (for example, the same endoscope as the endoscope 16) around a major axis in a case where the α loop is formed, data that can specify a rotation direction and a rotation angle of the insertion part of the endoscope around the major axis in a case where the inverse α loop is formed, or data that can specify a rotation direction and a rotation angle of the insertion part of the endoscope around the major axis in a case where the loop is not formed.

The lumen position information set 152A1 includes a plurality of pieces of lumen position information 152A1a (here, for example, three or more pieces of lumen position information 152A1a) arranged in time series. The lumen position information 152A1a is any one of first to third information. The first information is information that can specify a position of a lumen in a frame in a case where the α loop of the insertion part of the endoscope is formed in the endoscopy, in which the inside of the large intestine is imaged by the endoscope in a process until the α loop is formed, in each of a plurality of frames included in the endoscopic video image obtained by the imaging. The second information is information that can specify a position of a lumen in a frame in a case where the inverse α loop of the insertion part of the endoscope is formed in the endoscopy, in which the inside of the large intestine is imaged by the endoscope in a process until the inverse α loop is formed, in each of a plurality of frames included in the endoscopic video image obtained by the imaging. The third information is information that can specify a position of a lumen in a frame in a case where the loop of the insertion part of the endoscope is not formed in the endoscopy, in which the inside of the large intestine is imaged by the endoscope, in each of a plurality of frames included in the endoscopic video image obtained by the imaging.

The lumen position information 152A1a is information that can specify a position of any one of the eight divided regions 154 as a position where the lumen is shown. The eight divided regions 154 are obtained by dividing the frame included in the endoscopic video image into eight parts in the same manner as the eight divided regions 130A are obtained. The eight divided regions 154 are disposed at intervals of 45 degrees along the center C2 of the frame included in the endoscopic video image.

The lumen position information set 152A1 includes any one of first to third time-series information. The first time-series information is information in which the number of pieces of lumen position information 152A1a obtained from a time when the insertion part of the endoscope is inserted into the large intestine to a time when the α loop of the insertion part is formed in the endoscopy is arranged in time series. The first time-series information can also be said to be an accumulated result in which changes in the position of the lumen between a plurality of frames until the position of the lumen makes at least a half rotation counterclockwise around the eight divided regions 154 are accumulated.

The second time-series information is information in which the number of pieces of lumen position information 152A1a obtained from a time when the insertion part of the endoscope is inserted into the large intestine to a time when the inverse α loop of the insertion part is formed in the endoscopy is arranged in time series. The second time-series information can also be said to be an accumulated result in which changes in the position of the lumen between a plurality of frames until the position of the lumen makes at least a half rotation clockwise around the eight divided regions 154 are accumulated.

The third time-series information is information in which the number of pieces of lumen position information 152A1a obtained from a time when the insertion part of the endoscope is inserted into the large intestine to a case where the loop of the insertion part is not formed in the endoscopy is arranged in time series (for example, the number of frames corresponding to a statistical value such as an average value, a median value, a mode value, a maximum value, or a minimum value of the number of frames obtained until the α loop or the inverse α loop is formed).

Here, as the ground-truth data 152A2, data that can specify a rotation direction (for example, clockwise) and a rotation angle (for example, an angle of 180 degrees or more) of the insertion part of the endoscope around the major axis in a case where the α loop is formed is associated with the lumen position information set 152A1 including the first time-series information.

In addition, as the ground-truth data 152A2, data that can specify a rotation direction (for example, counterclockwise) and a rotation angle (for example, an angle of 180 degrees or more) of the insertion part of the endoscope around the major axis in a case where the inverse α loop is formed is associated with the lumen position information set 152A1 including the second time-series information.

Further, as the ground-truth data 152A2, data that can specify a rotation direction (for example, clockwise, counterclockwise, or neither clockwise nor counterclockwise) and a rotation angle (for example, an angle of less than 180 degrees) in a case where the loop is not formed is associated with the lumen position information set 152A1 including the third time-series information.

In the information processing device 100, the second learning execution unit 106C executes the machine learning using the dataset group 152. Hereinafter, the details will be described with reference to FIG. 13.

The second learning execution unit 106C executes processing using a model 156. Examples of the model 156 include the same neural network as the model 142 shown in FIG. 11. The second learning execution unit 106C acquires the dataset 152A from the dataset group 152. Then, the second learning execution unit 106C inputs the plurality of pieces of lumen position information 152A1a arranged in time series in the lumen position information set 152A1 included in the dataset 152A acquired from the dataset group 152 to the model 156 in time series. In a case where the plurality of pieces of lumen position information 152A1a arranged in time series are input, the model 156 performs inference and outputs an inference result 158. The second learning execution unit 106C calculates an error 160 between the inference result 158 and the ground-truth data 152A2 included in the dataset 152A acquired from the dataset group 152.

The second learning execution unit 106C calculates a plurality of adjustment values 162 that minimize the error 160. Then, the second learning execution unit 106C adjusts a plurality of optimization variables in the model 156 by using the plurality of adjustment values 162, to optimize the model 156. Examples of the plurality of optimization variables in the model 156 include a weight and a bias.

The second learning execution unit 106C repeatedly performs learning processing of inputting the lumen position information set 152A1 to the model 156, calculating the error 160, calculating the plurality of adjustment values 162, and adjusting the plurality of optimization variables in the model 156 using all the datasets 152A included in the dataset group 152. That is, the second learning execution unit 106C optimizes the model 156 by adjusting the plurality of optimization variables in the model 156 using the plurality of adjustment values 162 calculated to minimize the error 160 for each of all the lumen position information sets 152A1 included in all the datasets 152A. The rotation recognition model 94 is generated by optimizing the model 156 in this way. The rotation recognition model 94 is transmitted from the information processing device 100 to the medical support device 24 via the external I/Fs 80 and 104 (see FIG. 7), and is received by the medical support device 24. Then, in the medical support device 24, the rotation recognition model 94 is stored in the storage 86 by the processor 82 (see FIG. 6). The rotation recognition model 94 stored in the storage 86 is used by the recognition unit 82A (see FIG. 6).

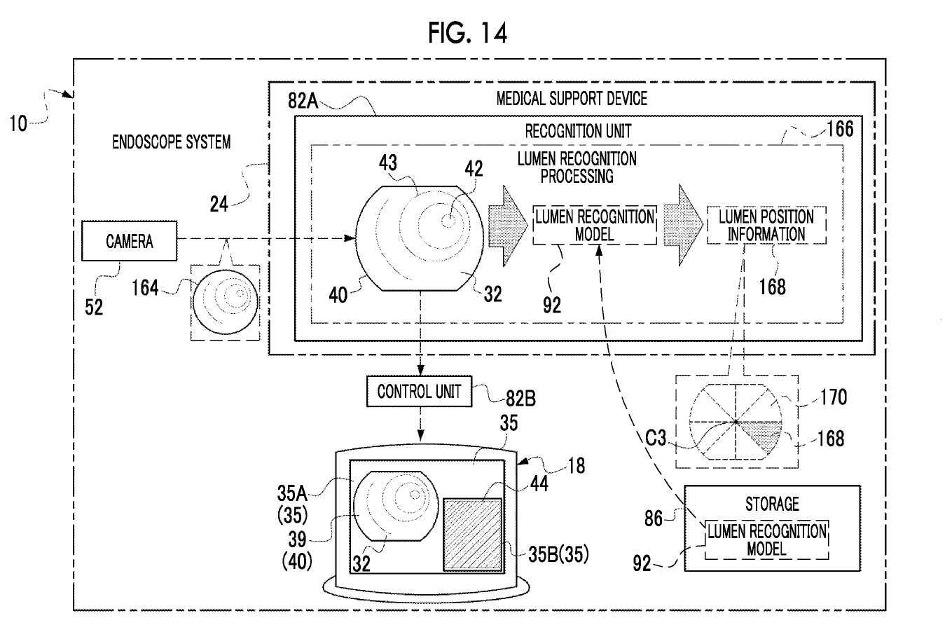

FIG. 14 shows an example of processing contents in the recognition unit 82A. As shown in FIG. 14, an image 164 obtained by imaging the intestinal wall 32 in the large intestine 28 including the lumen 42 with the camera 52 is acquired by the recognition unit 82A. The recognition unit 82A generates the frame 40 by executing various types of processing on the image 164. In the example shown in FIG. 14, the intestinal wall 32 having the folds 43 and the lumen 42 are shown in the frame 40.

The control unit 82B acquires the frame 40 from the recognition unit 82A and displays the acquired frame 40 in the first display region 35A.

The recognition unit 82A executes lumen recognition processing 166 on the frame 40. The lumen recognition processing 166 is processing of recognizing the lumen 42 shown in the frame 40 using the lumen recognition model 92 stored in the storage 86 (in other words, processing of specifying the existence position of the lumen 42, which is shown in the frame 40, in the frame 40 using the lumen recognition model 92).

The recognition unit 82A causes the lumen recognition model 92 to generate lumen position information 168 by inputting the frame 40 to the lumen recognition model 92. The lumen position information 168 is information indicating a position of the lumen 42 included in the large intestine 28 in the frame 40. The lumen position information 168 is information that can specify a position of any one of the eight divided regions 170 as a position where the lumen is shown. The eight divided regions 170 are obtained by dividing the frame 40 into eight parts in the same manner as the eight divided regions 130A are obtained. The eight divided regions 170 are disposed at intervals of 45 degrees along the center C3 of the frame 40. In the first embodiment, the lumen position information 168 is an example of “feature information” and “lumen position information” according to the present disclosure.

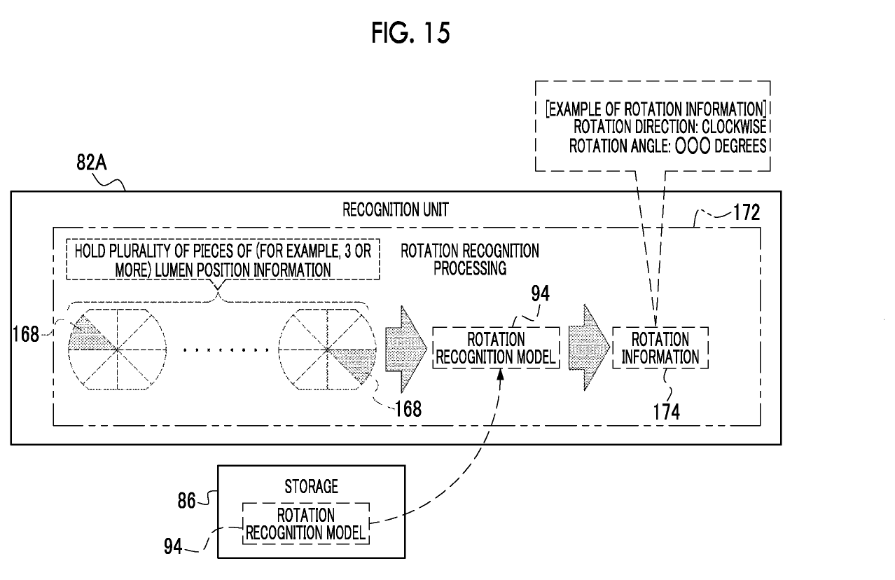

As shown in FIG. 15 as an example, the recognition unit 82A holds the plurality of pieces of lumen position information 168 in time series. Here, the plurality of pieces of lumen position information 168 in time series can also be said to be an accumulated result (for example, an integrated accumulated result) in which changes in the feature information (that is, information indicating a feature) obtained from the frame 40 between the plurality of frames 40 are accumulated. The recognition unit 82A generates rotation information 174 based on the accumulated result in which the changes in the feature information obtained from the frame 40 between the plurality of frames 40 are accumulated. Here, an example of the accumulated result in which the changes in the feature information obtained from the frame 40 between the plurality of frames 40 are accumulated includes the plurality of pieces of lumen position information 168 in time series obtained by executing the lumen recognition processing 166 (see FIG. 14) on each of the plurality of frames 40 obtained in time series by the recognition unit 82A.

The recognition unit 82A executes rotation recognition processing 172 on the plurality of pieces of lumen position information 168 in time series (here, for example, three or more pieces of lumen position information 168). The rotation recognition processing 172 is processing of recognizing the rotation information 174 from the plurality of pieces of lumen position information 168 in time series by using the rotation recognition model 94 stored in the storage 86 (in other words, processing of specifying a rotation direction and a rotation angle of the insertion part 48 around the major axis by using the rotation recognition model 94).

The recognition unit 82A causes the rotation recognition model 94 to generate the rotation information 174 by inputting the plurality of pieces of lumen position information 168 in time series to the rotation recognition model 94. In the first embodiment, the lumen recognition model 92 and the rotation recognition model 94 are examples of a “trained model” according to the present disclosure. In addition, in the first embodiment, the rotation information 174 is an example of “rotation information” according to the present disclosure.

The rotation information 174 includes information that can specify a rotation angle (for example, clockwise, counterclockwise, or neither clockwise nor counterclockwise) and a rotation direction of the insertion part 48 around the major axis. The rotation direction and the rotation angle of the insertion part 48 around the major axis refer to a relative rotation angle and a rotation direction of a second position with respect to a first position of the insertion part 48 around the major axis of the insertion part 48 or an absolute rotation angle and a rotation direction of the first position and the second position of the insertion part 48 around the major axis with respect to a reference angle (for example, an angle of a gravity direction recognized by AI-based or non-AI-based image recognition processing or a predetermined angle). Here, the first position is a reference position of the insertion part 48. Examples of the reference position of the insertion part 48 include a position of the insertion part 48 that is held by the doctor 12. The second position is a position that is a comparison target with the first position. Examples of the position that is a comparison target with the first position include the distal end surface 50A shown in FIG. 2.

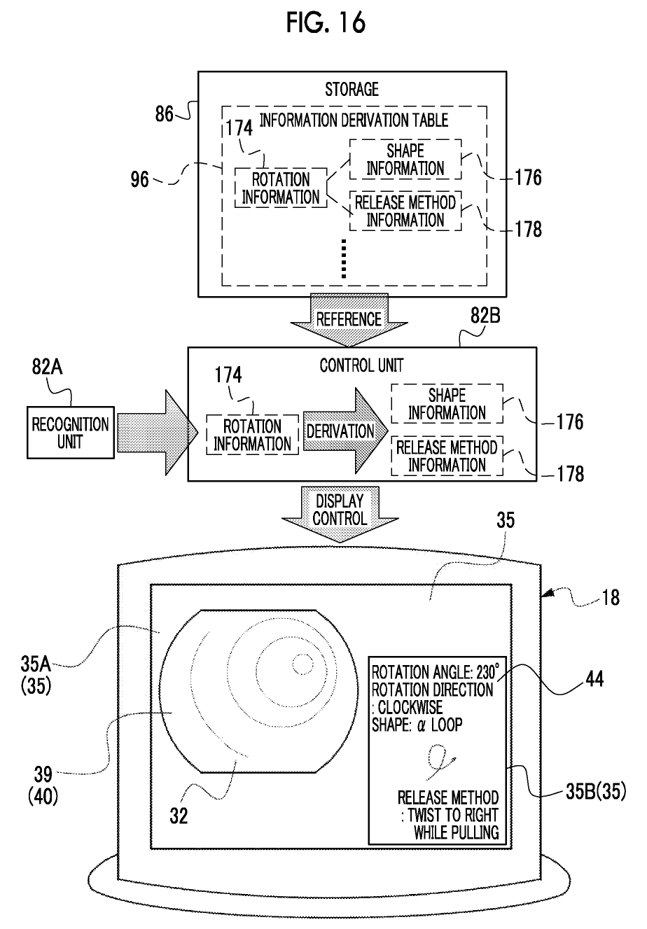

As shown in FIG. 16 as an example, the information derivation table 96 stored in the storage 86 is a table in which the rotation information 174 and shape information 176 and release method information 178 corresponding to the rotation information 174 are associated with each other. The shape information 176 is information representing the shape (for example, an α loop, an inverse α loop, or a non-loop) of the insertion part 48 in the large intestine 28. For example, the shape information 176 in a case where the distal end position of the insertion part 48 is rotated by 180 degrees or more with respect to the base end position of the insertion part 48 (for example, a position of the insertion part 48 that is held by the doctor 12) is information indicating that the insertion part 48 forms a loop. The shape information 176 in a case where the distal end position of the insertion part 48 is rotated by 180 degrees or more with respect to the base end position of the insertion part 48 includes loop classification information that classifies the shape of the loop based on the rotation direction specified from the rotation information 174. Examples of the loop classification information include information that classifies the loop of the insertion part 48 into an α loop and an inverse α loop based on the rotation direction specified from the rotation information 174.

More specifically, the shape information 176 in a case where the distal end position of the insertion part 48 is rotated clockwise by 180 degrees or more around the major axis of the insertion part 48 with respect to the base end position of the insertion part 48 is information indicating that the insertion part 48 forms an α loop. In addition, the shape information 176 in a case where the distal end position of the insertion part 48 is rotated counterclockwise by 180 degrees or more around the major axis of the insertion part 48 with respect to the base end position of the insertion part 48 is information indicating that the insertion part 48 forms an α loop. Further, the shape information 176 in a case where the distal end position of the insertion part 48 is not rotated by 180 degrees or more with respect to the base end position of the insertion part 48 is information indicating that the insertion part 48 does not form a loop.

The release method information 178 is information on a method of releasing the loop in a case where the shape represented by the shape information 176 associated with the rotation information 174 is the α loop or the inverse α loop.

The control unit 82B acquires the rotation information 174 from the recognition unit 82A. Then, the control unit 82B generates the shape information 176 and the release method information 178 based on the rotation information 174 from the recognition unit 82A. In the example shown in FIG. 16, the control unit 82B derives the shape information 176 and the release method information 178 corresponding to the rotation information 174 from the information derivation table 96 by referring to the information derivation table 96 stored in the storage 86. In the first embodiment, the shape information 176 is an example of “shape information” according to the present disclosure, and the release method information 178 is an example of “information on a method of releasing a loop” and “information based on the shape information” according to the present disclosure.

It should be noted that, in the example shown in FIG. 16, the form example has been described in which the release method information 178 is directly derived from the rotation information 174, but this is merely an example, and the shape information 176 may be derived from the rotation information 174 first, and then the release method information 178 may be derived from the shape information 176. In this case, for example, first, the control unit 82B derives the shape information 176 corresponding to the rotation information 174 from a first table in which the rotation information 174 and the shape information 176 are associated with each other by referring to the first table. Then, the control unit 82B derives the release method information 178 corresponding to the derived shape information 176 from a second table in which the shape information 176 and the release method information 178 are associated with each other by referring to the second table. In addition, the release method information 178 may be derived from both the rotation information 174 and the shape information 176. In this case, the control unit 82B derives the release method information 178 corresponding to the derived rotation information 174 and shape information 176 from a third table in which the rotation information 174 and the shape information 176 and the release method information 178 are associated with each other by referring to the third table.

The control unit 82B performs display control on the display device 18 based on the rotation information 174, the shape information 176, the release method information 178, and the like. That is, the control unit 82B displays the rotation information 174 acquired from the recognition unit 82A in the second display region 35B as visible information, and displays the shape information 176 and the release method information 178 derived from the information derivation table 96 in the second display region 35B as visible information. In the example shown in FIG. 16, in the second display region 35B, a rotation angle and a rotation direction specified by the rotation information 174 are displayed as a part of the auxiliary information 44 in text. In addition, in the second display region 35B, a type of the shape represented by the shape information 176 is displayed as a part of the auxiliary information 44 in text and a schematic diagram. Further, in the second display region 35B, a release method indicated by the release method information 178 is displayed as a part of the auxiliary information 44 in text.

It should be noted that, here, the visible display using the display device 18 is shown as an example of the output of the rotation information 174, the shape information 176, and the release method information 178, but this is merely an example. For example, the rotation information 174, the shape information 176, and the release method information 178 may be output as audible information in a voice, may be stored in a storage medium (for example, a memory and/or a magnetic tape), or may be recorded on a medium by a printer.

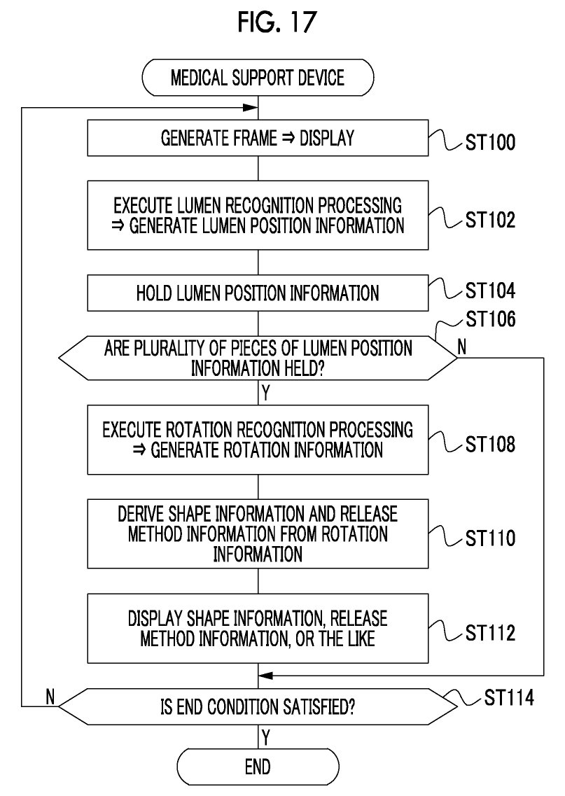

Next, an example of a flow of medical support processing performed by the endoscope system 10 will be described with reference to FIG. 17. A flow of the medical support processing shown in FIG. 17 is an example of a "medical support method" according to the present disclosure.

In the medical support processing shown in FIG. 17, first, in step ST100, the recognition unit 82A acquires the image 164 from the camera 52, and performs various types of processing on the acquired image 164 to generate the frame 40 (see FIG. 14). Then, the control unit 82B displays the latest frame 40 generated by the recognition unit 82A in the first display region 35A. After the processing in step ST100 is executed, the medical support processing proceeds to step ST102.

In step ST102, the recognition unit 82A causes the lumen recognition model 92 to generate the lumen position information 168 by executing the lumen recognition processing 166 using the lumen recognition model 92 (see FIG. 14). After the processing in step ST102 is executed, the medical support processing proceeds to step ST104.

In step ST104, the recognition unit 82A holds the lumen position information 168 generated in step ST102 in time series. After the processing in step ST104 is executed, the medical support processing proceeds to step ST106.

In step ST106, the recognition unit 82A determines whether or not a state in which the plurality of pieces of lumen position information 168 (here, for example, three or more pieces of lumen position information 168) are held in time series is established. In step ST106, in a case where the state in which the plurality of pieces of lumen position information are held in time series is not established (for example, in a case where only one piece of lumen position information 168 is held), a negative determination is made, and the medical support processing proceeds to step ST114. In step ST106, in a case where the state in which the plurality of pieces of lumen position information are held in time series is established, an affirmative determination is made, and the medical support processing proceeds to step ST108.

In step ST108, the recognition unit 82A causes the rotation recognition model 94 to generate the rotation information 174 by executing the rotation recognition processing 172 on the plurality of pieces of lumen position information 168 held in time series (see FIG. 15). After the processing in step ST108 is executed, the medical support processing proceeds to step ST110.

In step ST110, the control unit 82B derives the shape information 176 and the release method information 178 corresponding to the rotation information 174 generated in step ST108 by referring to the information derivation table 96 (see FIG. 16). After the processing in step ST110 is executed, the medical support processing proceeds to step ST112.

In step ST112, the control unit 82B displays the shape information 176 and the release method information 178 derived in step ST110 and the rotation information 174 generated in step ST108 in the second display region 35B as the auxiliary information 44 that is visualized (see FIG. 16). After the processing in step ST112 is executed, the medical support processing proceeds to step ST114.

In step ST114, the control unit 82B determines whether or not a medical support processing end condition is satisfied. Examples of the medical support processing end condition include a condition in which an instruction to end the medical support processing is issued to the endoscope system 10 (for example, a condition in which the reception device 64 receives the instruction to end the medical support processing).

In step ST114, in a case in which the medical support processing end condition is not satisfied, a negative determination is made, and the medical support processing proceeds to step ST100 shown in FIG. 17. In a case in which the medical support processing end condition is satisfied in step ST100, an affirmative determination is made, and the medical support processing ends.

As described above, in the endoscope system 10, the plurality of frames 40 obtained by imaging the inside of the large intestine 28 by the endoscope 16 in which the insertion part 48 is inserted into the large intestine 28 are input to the lumen recognition model 92, and the lumen position information 168 is generated by the lumen recognition model 92. In addition, the plurality of pieces of lumen position information 168 in time series are input to the rotation recognition model 94, and the rotation information 174 is generated by the rotation recognition model 94. Then, the shape information 176 is derived based on the rotation information 174. Therefore, it is possible to estimate the shape of the insertion part 48 in a case where the insertion part 48 of the endoscope 16 is inserted into the large intestine 28 without using an external device that recognizes the shape of the insertion part 48 in the case where the insertion part 48 of the endoscope 16 is inserted into the large intestine 28.

In addition, in the endoscope system 10, the rotation information 174 is obtained based on the accumulated result in which the changes in the feature information (that is, information indicating a feature) obtained from the frame 40 between the plurality of frames 40 are accumulated. Here, examples of the feature information include the lumen position information 168 (see FIG. 15). Therefore, it is possible to generate the accurate shape information 176 by using the cumulative change in the position of the lumen 42 shown in the frame 40. In addition, the rotation information 174 can be easily obtained as compared to a case where a dedicated sensor for obtaining the rotation information 174 is used or a dedicated device for obtaining the rotation information 174 is used outside the endoscope system 10.

In addition, in the endoscope system 10, information on a relative rotation angle and a rotation direction of a second position with respect to a first position of the insertion part 48 around the major axis of the insertion part 48 or information on an absolute rotation angle and a rotation direction of the first position and the second position of the insertion part 48 around the major axis with respect to a reference angle is used as the rotation information 174. Therefore, it is possible to accurately estimate the shape of the insertion part 48 by using the relative or absolute rotation information 174 of the plurality of positions of the insertion part 48.

In addition, in the endoscope system 10, the shape information 176 in a case where the distal end position of the insertion part 48 is rotated by 180 degrees or more with respect to the base end position of the insertion part 48 includes information indicating that the insertion part 48 forms a loop. Therefore, the doctor 12 can accurately estimate that the insertion part 48 forms a loop by referring to the shape information 176.

In addition, in the endoscope system 10, the shape information 176 in a case where the distal end position of the insertion part 48 is rotated by 180 degrees or more with respect to the base end position of the insertion part 48 includes loop classification information that classifies the shape of the loop based on the rotation direction specified from the rotation information 174. Therefore, the doctor 12 can accurately estimate the type of the shape of the loop of the insertion part 48 by referring to the shape information 176.

Here, the loop classification information is information that classifies the loop of the insertion part 48 into the α loop and the inverse α loop based on the rotation direction specified from the rotation information 174. Therefore, the doctor 12 can accurately estimate whether the type of the shape of the loop of the insertion part 48 is the α loop or the inverse α loop by referring to the shape information 176.

In addition, in the endoscope system 10, the release method information 178 is output based on the rotation information 174. The release method information 178 is information on a method of releasing the loop of the insertion part 48. Therefore, it is possible to support the doctor 12 to select an appropriate operation for releasing the loop of the insertion part 48.

[Second Embodiment] In the first embodiment, the form example (see FIG. 15) has been described in which the rotation information 174 is obtained based on the accumulated result in which the changes in the lumen position information 168 between the plurality of frames 40 are accumulated, but this is merely an example. In the second embodiment, a form example will be described in which the rotation information 174 is obtained based on information other than the accumulated result in which the changes in the lumen position information 168 between the plurality of frames 40 are accumulated. In the second embodiment, constituents described in the first embodiment will be designated by the same reference numerals and will not be described, and different parts from the first embodiment will be described.

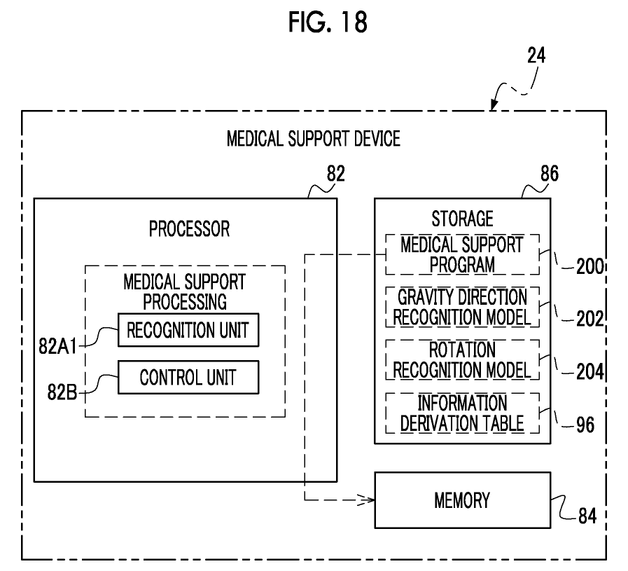

FIG. 18 is a block diagram showing an example of functions of main units of the processor 82 included in the medical support device 24 and an example of information stored in the storage 86 according to a second embodiment. As shown in FIG. 18, a medical support program 200 is stored in the storage 86. The medical support program 200 in the second embodiment is an example of a "program" according to the present disclosure.

The processor 82 reads out the medical support program 200 from the storage 86, and executes the readout medical support program 200 on the memory 84 to perform medical support processing. The medical support processing is implemented by the processor 82 operating as a recognition unit 82A1 and a control unit 82B in accordance with the medical support program 200 executed on the memory 84.

The storage 86 stores a gravity direction recognition model 202, a rotation recognition model 204, and the information derivation table 96. Although the details will be described later, each of the gravity direction recognition model 202 and the rotation recognition model 204 is a machine learning model and is used by the recognition unit 82A1. Examples of the machine learning model include a neural network (for example, a recurrent neural network, a two-dimensional convolutional neural network, and/or a three-dimensional convolutional neural network).

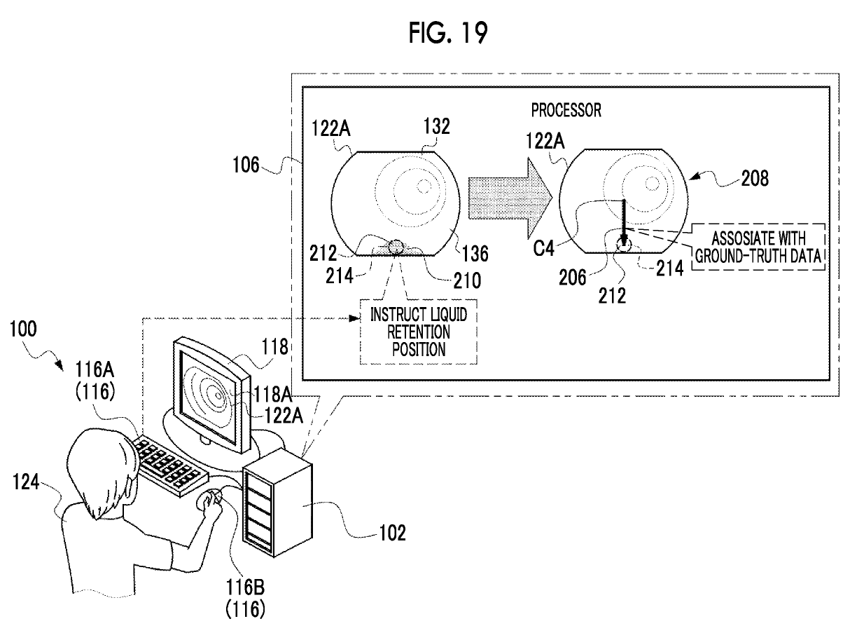

FIG. 19 is a conceptual diagram showing an example of a method of generating training data 208 by the processor 106 of the information processing device 100 associating ground-truth data 206 with the example image 122A.

As shown in FIG. 19, in a state where the example image 122A is displayed on the screen 118A, the annotator 124 issues an instruction to the processor 106 to indicate a liquid retention position 212, which is a position of a liquid 210 that is retained on the wall of the intestinal tract 136 in the large intestine 132 shown in the example image 122A in the example image 122A, via the reception device 116. The processor 106 displays a circular frame 214 in a superimposed manner at a position surrounding the liquid retention position 212 on the example image 122A in accordance with the instruction received by the reception device 116. The frame 214 is a mark that defines the liquid retention position 212 in the example image 122A. Here, the shape of the frame 214 is a circular shape, but the shape may be other than the circular shape. The size of the frame 214 can be changed in response to the instruction received by the reception device 116.

The annotator 124 issues a determination instruction, which is an instruction to determine the liquid retention position 212, to the processor 106 via the reception device 116 in a state where the frame 214 is arranged at the position surrounding the liquid retention position 212. As a result, the processor 106 determines the liquid retention position 212.

The processor 106 generates the training data 208 by associating the ground-truth data 206, which is information that can specify the gravity direction in the example image 122A, with the example image 122A. The ground-truth data 206 is a vector having a center C4 of the example image 122A as a start point and a center of the liquid retention position 212 as an end point.

In this way, the processor 106 generates a plurality of pieces of training data 208 by repeatedly performing processing of associating the ground-truth data 206 with each of the example images 122A included in the example image set 122 in accordance with the instruction given from the annotator 124.

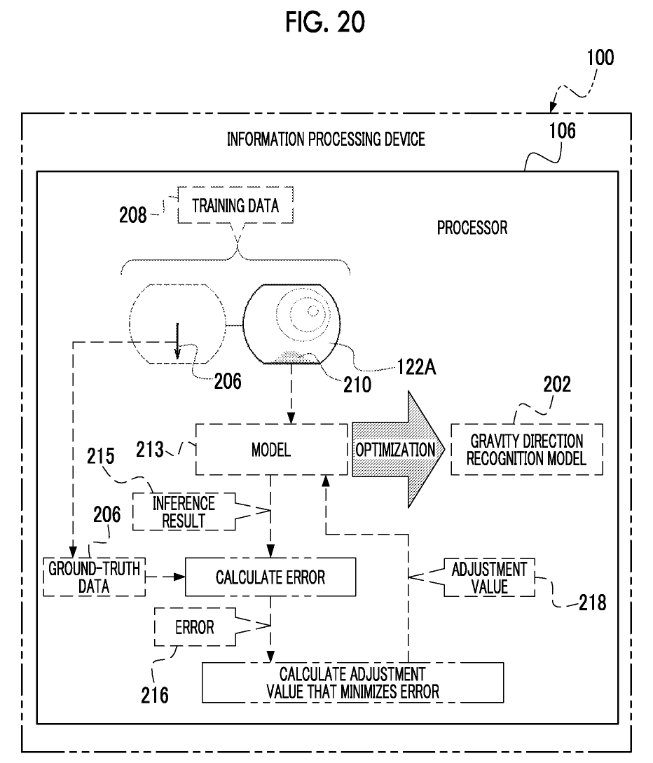

FIG. 20 is a conceptual diagram showing an example of an aspect in which the gravity direction recognition model 202 is generated by performing machine learning using the training data 208 by the processor 106 in the information processing device 100.

As shown in FIG. 20, in the information processing device 100, the processor 106 executes the machine learning using the training data 208 generated in the above-described manner. Hereinafter, the details will be described with reference to FIG. 20.

In the example shown in FIG. 20, the processor 106 executes processing using a model 213. Examples of the model 213 include the neural network exemplified in the first embodiment. The processor 106 inputs the example image 122A included in the training data 208 to the model 213. In a case in which the example image 122A is input, the model 213 performs an inference to output an inference result 215. The processor 106 calculates an error 216 between the inference result 215 and the ground-truth data 206 included in the training data 208.

The processor 106 calculates a plurality of adjustment values 218 that minimize the error 216. Then, the processor 106 optimizes the model 213 by adjusting a plurality of optimization variables in the model 213 using the plurality of adjustment values 218. Examples of the plurality of optimization variables in the model 213 include a weight and a bias.

The processor 106 repeatedly performs learning processing of inputting the example image 122A included in the training data 208 to the model 213, calculating the error 216, calculating the plurality of adjustment values 218, and adjusting the plurality of optimization variables in the model 213 using the plurality of pieces of training data 208. That is, the processor 106 adjusts the plurality of optimization variables in the model 213 using the plurality of adjustment values 218 calculated such that the error 216 is minimized for each of the plurality of example images 122A included in the plurality of pieces of training data 208, to optimize the model 213. The gravity direction recognition model 202 is generated by optimizing the model 213 in this way. The gravity direction recognition model 202 is transmitted from the information processing device 100 to the medical support device 24 via the external I/Fs 80 and 104 (see FIG. 7), and is received by the medical support device 24. Then, in the medical support device 24, the processor 82 stores the gravity direction recognition model 202 in the storage 86 (see FIG. 18). The gravity direction recognition model 202 stored in the storage 86 is used by the recognition unit 82A1 (see FIG. 18).

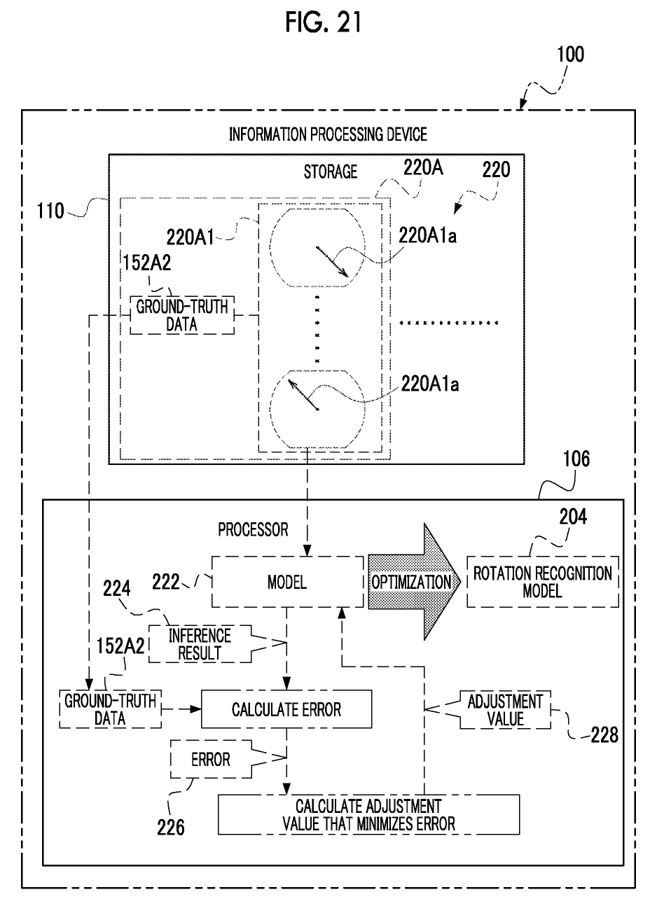

As shown in FIG. 21, a dataset group 220 is stored in the storage 110 of the information processing device 100. The dataset group 220 is a set of a plurality of datasets 220A. The plurality of datasets 220A have different contents. The dataset 220A is training data in which a gravity direction information set 220A1 and ground-truth data 152A2 described in the first embodiment are associated with each other. The dataset 220A is generated by associating the ground-truth data 152A2 with the gravity direction information set 220A1 in the same manner as the training data 128 shown in FIG. 8 described in the first embodiment is generated by the training data generation unit 106A.

The gravity direction information set 220A1 includes a plurality of pieces of gravity direction information 220A1a (here, for example, three or more pieces of gravity direction information 220A1a) arranged in time series. The gravity direction information 220A1a is any one of fourth to sixth information. The fourth information is information (for example, a direction vector) indicating a gravity direction that can be specified from each of a plurality of frames included in the endoscopic video image obtained by imaging the inside of the large intestine with the endoscope in a process until the α loop of the insertion part of the endoscope is formed in the endoscopy. The fifth information is information (for example, a direction vector) indicating a gravity direction that can be specified from each of a plurality of frames included in the endoscopic video image obtained by imaging the inside of the large intestine with the endoscope in a process until the inverse α loop of the insertion part of the endoscope is formed in the endoscopy. The sixth information is information (for example, a direction vector) indicating a gravity direction that can be specified from each of a plurality of frames included in the endoscopic video image obtained by imaging the inside of the large intestine with the endoscope in a case where the loop of the insertion part of the endoscope is not formed in the endoscopy.

The gravity direction information set 220A1 includes any one of fourth to sixth time-series information. The fourth time-series information is information in which the number of pieces of gravity direction information 220A1a obtained from a time when the insertion part of the endoscope is inserted into the large intestine to a time when the α loop of the insertion part is formed in the endoscopy is arranged in time series. The fourth time-series information can also be said to be an accumulated result in which changes in the gravity direction between a plurality of frames until the gravity direction makes at least a half rotation counterclockwise are accumulated.

The fifth time-series information is information in which the number of pieces of gravity direction information 220A1a obtained from a time when the insertion part of the endoscope is inserted into the large intestine to a time when the inverse α loop of the insertion part is formed in the endoscopy is arranged in time series. The fifth time-series information can also be said to be an accumulated result in which changes in the gravity direction between a plurality of frames until the gravity direction makes at least a half rotation clockwise are accumulated.

The sixth time-series information is information in which the number of pieces of gravity direction information 220A1a obtained from a time when the insertion part of the endoscope is inserted into the large intestine to a case where the loop of the insertion part is not formed in the endoscopy is arranged in time series (for example, the number of frames corresponding to a statistical value such as an average value, a median value, a mode value, a maximum value, or a minimum value of the number of frames obtained until the α loop or the inverse α loop is formed).

Here, as the ground-truth data 152A2, data that can specify a rotation direction (for example, clockwise) and a rotation angle (for example, an angle of 180 degrees or more) of the insertion part of the endoscope around the major axis in a case where the α loop is formed is associated with the gravity direction information set 220A1 including the fourth time-series information.

In addition, as the ground-truth data 152A2, data that can specify a rotation direction (for example, counterclockwise) and a rotation angle (for example, an angle of 180 degrees or more) of the insertion part of the endoscope around the major axis in a case where the inverse α loop is formed is associated with the gravity direction information set 220A1 including the fifth time-series information.

Further, as the ground-truth data 152A2, data that can specify a rotation direction (for example, clockwise, counterclockwise, or neither clockwise nor counterclockwise) and a rotation angle (for example, an angle of less than 180 degrees) in a case where the loop is not formed is associated with the gravity direction information set 220A1 including the sixth time-series information.

In the information processing device 100, the processor 106 executes the machine learning using the dataset group 220. Hereinafter, the details will be described with reference to FIG. 21.

The processor 106 executes processing using a model 222. Examples of the model 222 include the neural network exemplified in the first embodiment. The processor 106 acquires the dataset 220A from the dataset group 220. Then, the processor 106 inputs the plurality of pieces of gravity direction information 220A1a arranged in time series in the gravity direction information set 220A1 included in the dataset 220A acquired from the dataset group 220 to the model 222 in time series. In a case where the plurality of pieces of gravity direction information 220A1a arranged in time series are input, the model 222 performs inference and outputs an inference result 224. The processor 106 calculates an error 226 between the inference result 224 and the ground-truth data 152A2 included in the dataset 220A acquired from the dataset group 220.

The processor 106 calculates a plurality of adjustment values 228 that minimize the error 226. Then, the processor 106 optimizes the model 222 by adjusting a plurality of optimization variables in the model 222 using the plurality of adjustment values 228. Examples of the plurality of optimization variables in the model 222 include a weight and a bias.

The processor 106 repeatedly performs learning processing of inputting the gravity direction information set 220A1 to the model 222, calculating the error 226, calculating the plurality of adjustment values 228, and adjusting the plurality of optimization variables in the model 222 using all the datasets 220A included in the dataset group 220. That is, the processor 106 optimizes the model 222 by adjusting the plurality of optimization variables in the model 222 using the plurality of adjustment values 228 calculated to minimize the error 226 for each of all the gravity direction information sets 220A1 included in all the datasets 220A. The rotation recognition model 204 is generated by optimizing the model 222 in this way. The rotation recognition model 204 is transmitted from the information processing device 100 to the medical support device 24 via the external I/Fs 80 and 104 (see FIG. 7), and is received by the medical support device 24. Then, in the medical support device 24, the rotation recognition model 204 is stored in the storage 86 by the processor 82 (see FIG. 18). The rotation recognition model 204 stored in the storage 86 is used by the recognition unit 82A1 (see FIG. 18).

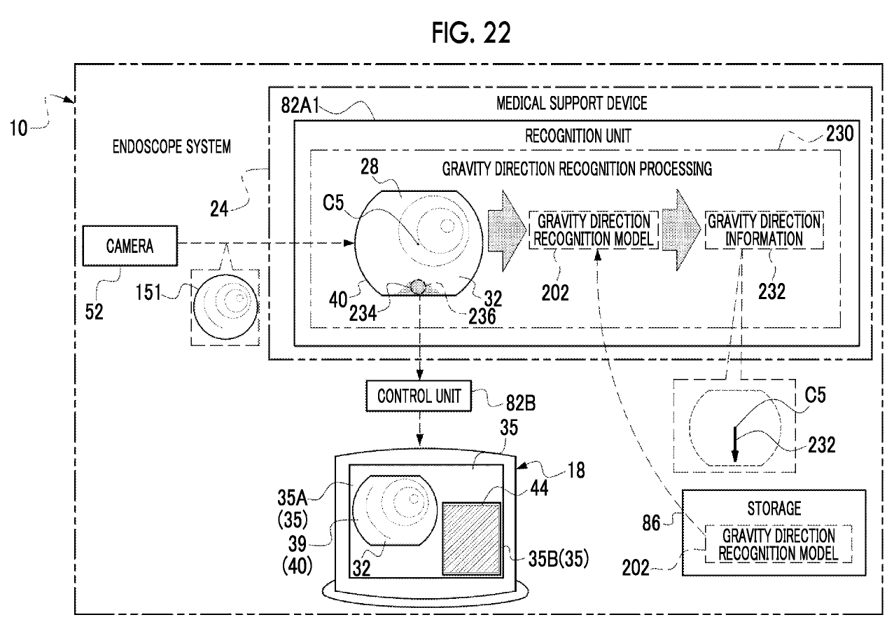

FIG. 22 shows an example of processing contents in the recognition unit 82A1. The recognition unit 82A1 is different from the recognition unit 82A described in the first embodiment in that the gravity direction recognition processing 230 is executed on the frame 40 instead of the lumen recognition processing 153.

The gravity direction recognition processing 230 is processing of recognizing the gravity direction in the large intestine 28 shown in the frame 40 by using the gravity direction recognition model 202 stored in the storage 86 (in other words, processing of specifying the gravity direction in the large intestine 28 shown in the frame 40 by using the gravity direction recognition model 202).

The recognition unit 82A1 causes the gravity direction recognition model 202 to generate gravity direction information 232 by inputting the frame 40 to the gravity direction recognition model 202. The gravity direction information 232 is information that can specify the gravity direction in the large intestine 28. Examples of the gravity direction information 232 include a vector having a center C5 of the frame 40 as a start point and a center of a liquid retention position 236 (here, for example, a position of a circular frame surrounding a center of a liquid 234) that is a position of a liquid 234 in the frame 40, which is retained on the intestinal wall 32 of the large intestine 28 shown in the frame 40 as an end point. In the second embodiment, the gravity direction information 232 is an example of “feature information” and “gravity direction information” according to the present disclosure.

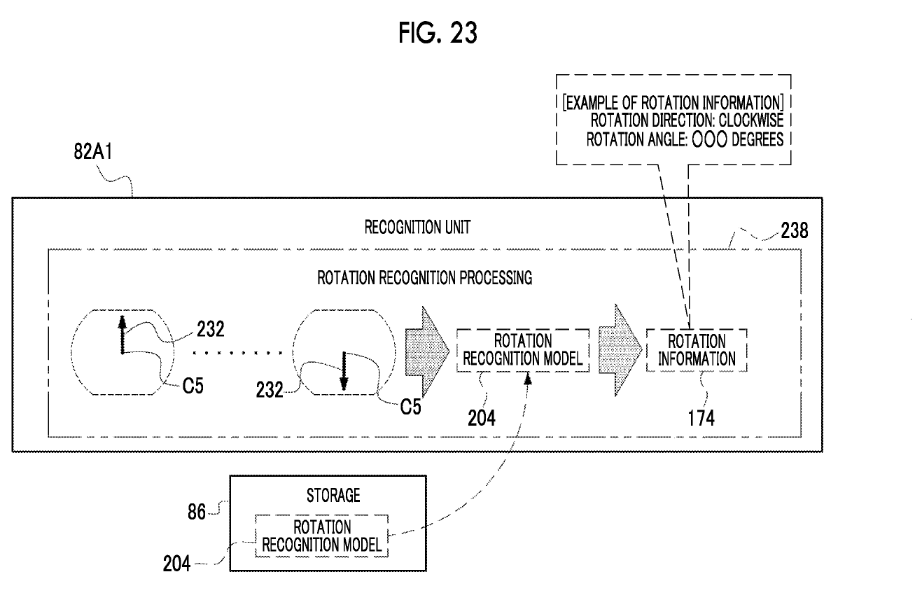

As shown in FIG. 23 as an example, the recognition unit 82A1 holds the plurality of pieces of gravity direction information 232 in time series. Here, the plurality of pieces of gravity direction information 232 in time series can also be said to be an accumulated result in which the changes in the feature information (that is, information indicating a feature) obtained from the frame 40 between the plurality of frames 40 are accumulated. As in the recognition unit 82A described in the first embodiment, the recognition unit 82A1 generates the rotation information 174 based on the accumulated result in which the changes in the feature information obtained from the frame 40 between the plurality of frames 40 are accumulated. Here, an example of the accumulated result in which the changes in the feature information obtained from the frame 40 between the plurality of frames 40 are accumulated includes the plurality of pieces of gravity direction information 232 in time series obtained by executing the gravity direction recognition processing 230 (see FIG. 22) on each of the plurality of frames 40 obtained in time series by the recognition unit 82A1.

The recognition unit 82A1 executes rotation recognition processing 238 on the plurality of pieces of gravity direction information 232 in time series (here, for example, three or more pieces of gravity direction information 232). The rotation recognition processing 238 is processing of recognizing the rotation information 174 from the plurality of pieces of gravity direction information 232 in time series by using the rotation recognition model 204 stored in the storage 86 (in other words, processing of specifying a rotation direction and a rotation angle of the insertion part 48 around the major axis by using the rotation recognition model 204).

The recognition unit 82A1 causes the rotation recognition model 204 to generate the rotation information 174 by inputting the plurality of pieces of gravity direction information 232 in time series to the rotation recognition model 204. In the second embodiment, the gravity direction recognition model 202 and the rotation recognition model 204 are examples of a “trained model” according to the present disclosure. In addition, in the second embodiment, the rotation information 174 is an example of “rotation information” according to the present disclosure.

The rotation information 174 obtained in this way is used by the control unit 82B in the same manner as in the first embodiment. As a result, the same effects as those of the above-described first embodiment can be obtained.

[Third Embodiment] In the first embodiment, the form example (see FIG. 15) has been described in which the rotation information 174 is obtained based on the accumulated result in which the changes in the lumen position information 168 between the plurality of frames 40 are accumulated, and in the second embodiment, the form example (see FIG. 23) has been described in which the rotation information 174 is obtained based on the accumulated result in which the changes in the gravity direction information 232 between the plurality of frames 40 are accumulated, but the present disclosure is not limited thereto. In the third embodiment, a form example will be described in which the rotation information 174 is obtained based on information other than the accumulated result in which the changes in the lumen position information 168 between the plurality of frames 40 are accumulated and the accumulated result in which the changes in the gravity direction information 232 between the plurality of frames 40 are accumulated. In the third embodiment, constituents described in the first and second embodiment will be designated by the same reference numerals and will not be described, and different parts from the first and second embodiment will be described.

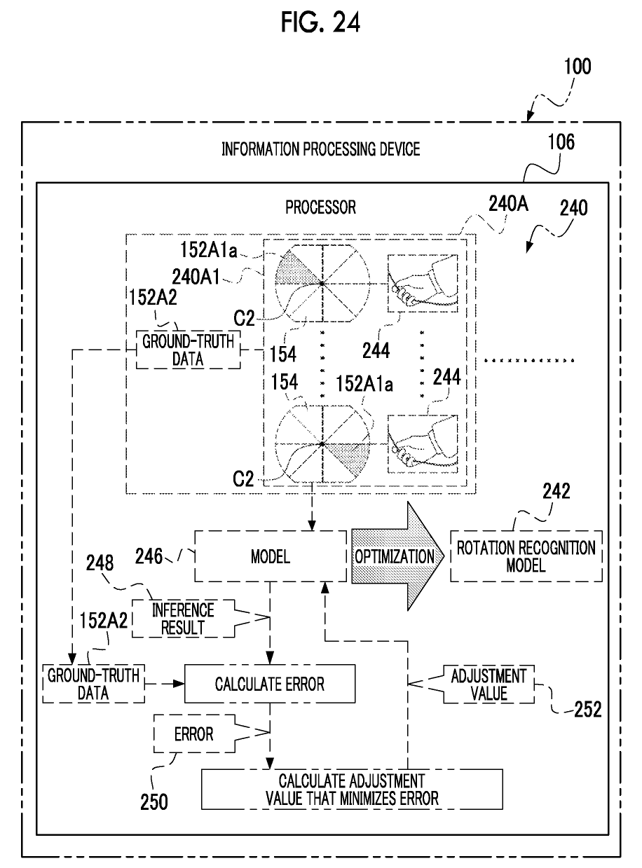

FIG. 24 is a conceptual diagram showing an example of an aspect in which the rotation recognition model 242 is generated by performing machine learning using a dataset group 240 by the processor 106 in the information processing device 100.

As shown in FIG. 24, the dataset group 240 is a set of a plurality of datasets 240A. The plurality of datasets 240A have different contents. The dataset 240A is training data in which an example data set 240A1 and the ground-truth data 152A2 are associated with each other. The dataset 240A is generated by associating the ground-truth data 152A2 with the example data set 240A1 in the same manner as the training data 128 shown in FIG. 8 is generated by the training data generation unit 106A.

The example data set 240A1 includes a plurality of pieces of lumen position information 152A1a (here, for example, three or more pieces of lumen position information 152A1a) arranged in time series and a plurality of hand images 244 (here, for example, three or more hand images 244) arranged in time series. One hand image 244 is associated with one piece of lumen position information 152A1a.

The hand image 244 is an image obtained by imaging a region including a hand-held part of the doctor (that is, a doctor who operates the insertion part of the endoscope) in the insertion part of the endoscope by the camera. In addition to the doctor's hand, a portion of the insertion part of the endoscope that is held by the doctor is also shown in the hand image 244. The hand image 244 is an image obtained by imaging a region including a hand-held part of the doctor in the insertion part of the endoscope (that is, a site of the doctor who operates the insertion part of the endoscope) in a case of imaging for obtaining the example image 122A used for generating the lumen position information 152A1a (that is, the lumen position information 152A1a forming a pair with the hand image 244) associated with the hand image 244. That is, the lumen position information 152A1a and the hand image 244 in a relationship of being associated with each other are obtained at the same timing.

In the information processing device 100, the processor 106 executes the machine learning using the dataset group 240. Hereinafter, the details will be described with reference to FIG. 24.

The processor 106 executes processing using a model 246. Examples of the model 246 include the same neural network as the model 142 shown in FIG. 11. The processor 106 acquires the dataset 240A from the dataset group 240. Then, the processor 106 inputs each of the plurality of pieces of lumen position information 152A1a arranged in time series in the example data set 240A1 included in the dataset 240A acquired from the dataset group 240 and each of the hand images 244 associated with each piece of lumen position information 152A1a to the model 246 in time series. In a case where the plurality of pieces of lumen position information 152A1a arranged in time series and the plurality of hand images 244 arranged in time series are input, the model 246 performs inference and outputs an inference result 248. The processor 106 calculates an error 250 between the inference result 248 and the ground-truth data 152A2 included in the dataset 240A acquired from the dataset group 240.

The processor 106 calculates a plurality of adjustment values 252 that minimize the error 250. Then, the processor 106 optimizes the model 246 by adjusting a plurality of optimization variables in the model 246 using the plurality of adjustment values 252. Examples of the plurality of optimization variables in the model 246 include a weight and a bias.

The processor 106 repeatedly performs learning processing of inputting the example data set 240A1 to the model 246, calculating the error 250, calculating the plurality of adjustment values 252, and adjusting the plurality of optimization variables in the model 246 using all the datasets 240A included in the dataset group 240. That is, the processor 106 optimizes the model 246 by adjusting the plurality of optimization variables in the model 246 using the plurality of adjustment values 252 calculated to minimize the error 250 for each set (each pair) of all the lumen position information sets 152A1 and all the hand images 244 included in all the datasets 240A. The rotation recognition model 242 is generated by optimizing the model 246 in this way. The rotation recognition model 242 is transmitted from the information processing device 100 to the medical support device 24 via the external I/Fs 80 and 104 (see FIG. 7), and is received by the medical support device 24. Then, in the medical support device 24, the processor 82 stores the rotation recognition model 242 in the storage 86. The rotation recognition model 242 stored in the storage 86 is used by the processor 82.

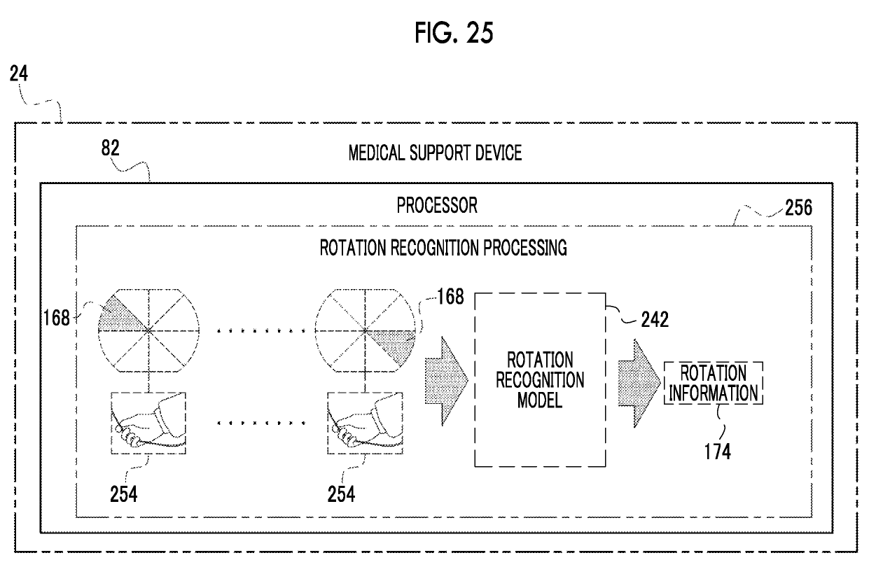

In this case, for example, as shown in FIG. 25, the processor 82 holds the plurality of pieces of lumen position information 168 in time series and a plurality of hand images 254 in time series. One hand image 254 is associated with one piece of lumen position information 168.

The hand image 254 is an image obtained by imaging a region including a hand-held part of the doctor 12 in the insertion part 48 of the endoscope 16 (that is, a hand-held part of the doctor 12 who operates the insertion part 48 of the endoscope 16) by the camera. In addition to the doctor 12's hand, a portion of the insertion part 48 of the endoscope 16 that is held by the doctor 12 is also shown in the hand image 254. The hand image 254 is an image obtained by imaging a region including a hand-held part of the doctor 12 in the insertion part 48 of the endoscope 16 (that is, a hand-held part of the doctor 12 who operates the insertion part 48 of the endoscope 16) in a case of imaging for obtaining the frame 40 used for generating the lumen position information 168 (that is, the lumen position information 168 forming a pair with the hand image 254) associated with the hand image 254. That is, the lumen position information 168 and the hand image 254 in a relationship of being associated with each other are obtained at the same timing.

Here, the set of the plurality of pieces of lumen position information 168 in time series and the plurality of hand images 254 in time series can also be said to be an accumulated result in which the changes in the feature information (that is, information indicating a feature) obtained from the frame 40 between the plurality of frames 40 are accumulated. The processor 82 generates the rotation information 174 based on the accumulated result in which the changes in the feature information obtained from the frame 40 between the plurality of frames 40 are accumulated.

The processor 82 executes rotation recognition processing 256 on a combination of the plurality of pieces of lumen position information 168 in time series (here, for example, three or more pieces of lumen position information 168) and the plurality of hand images 254 in time series. The rotation recognition processing 256 is processing of recognizing the rotation information 174 from the combination of the plurality of pieces of lumen position information 168 in time series and the plurality of hand images 254 in time series by using the rotation recognition model 242 stored in the storage 86 (that is, the rotation recognition model 242 obtained in the manner described using the example shown in FIG. 24) (in other words, processing of specifying a rotation direction and a rotation angle of the insertion part 48 around the major axis by using the rotation recognition model 242).

The processor 82 causes the rotation recognition model 242 to generate the rotation information 174 by inputting the combination of the plurality of pieces of lumen position information 168 in time series and the plurality of hand images 254 in time series to the rotation recognition model 242 in time series. The rotation information 174 obtained in this way is used by the control unit 82B in the same manner as in the first embodiment. As a result, the same effects as those of the above-described first embodiment can be obtained. In addition, since the rotation information 174 is estimated by the AI method using the plurality of pieces of lumen position information 168 in time series and the plurality of hand images 254 in time series, the high-accuracy rotation information 174 can be obtained as compared to a case where the rotation information 174 is estimated from only the plurality of pieces of lumen position information 168 in time series.

In the third embodiment, the lumen recognition model 92 and the rotation recognition model 242 are examples of a “trained model” according to the present disclosure. In addition, in the third embodiment, the rotation information 174 is an example of “rotation information” according to the present disclosure. In addition, in the third embodiment, the doctor 12 is an example of a “operator” according to the present disclosure, and the plurality of hand images 244 are examples of “a plurality of images” according to the present disclosure.

[Fourth Embodiment] In the first embodiment, the form example has been described in which the shape information 176 corresponding to the rotation information 174 is derived from the information derivation table 96, but this is merely an example. In the fourth embodiment, a form example will be described in which the shape information 176 corresponding to the rotation information 174 is derived by the AI method. In the fourth embodiment, constituents described in the first to third embodiment will be designated by the same reference numerals and will not be described, and different parts from the first to third embodiment will be described.

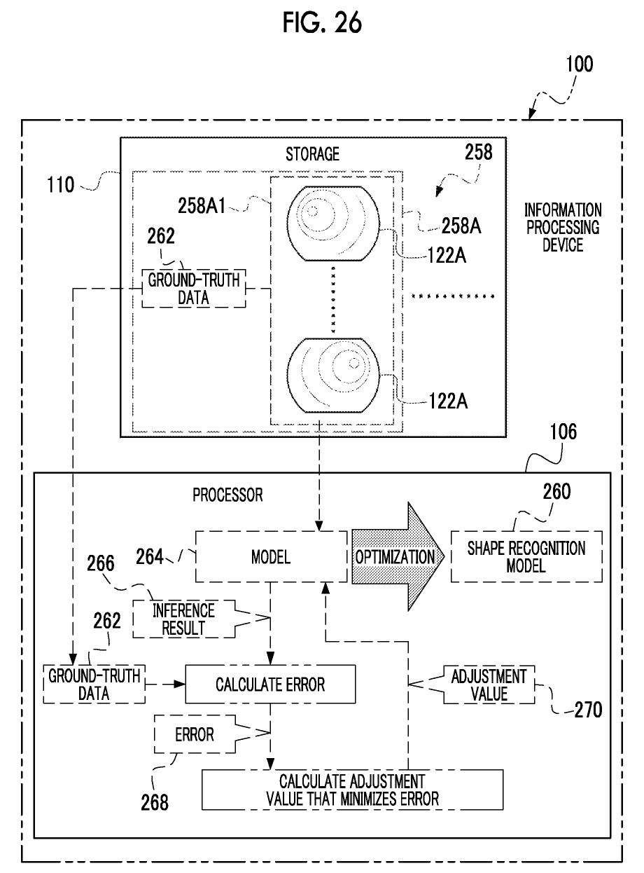

FIG. 26 is a conceptual diagram showing an example of an aspect in which the shape recognition model 260 is generated by performing machine learning using a dataset group 258 by the processor 106.

As shown in FIG. 26, the dataset group 258 is a set of a plurality of datasets 258A. The plurality of datasets 258A have different contents. The dataset 258A is training data in which an example image set 258A1 and ground-truth data 262 are associated with each other. The dataset 258A is generated by associating the ground-truth data 262 with the example image set 258A1 in the same manner as the training data 128 shown in FIG. 8 is generated by the training data generation unit 106A.

The ground-truth data 262 is data that can specify a shape (for example, an α loop, an inverse α loop, or a non-loop) of the insertion part of the endoscope in the large intestine. As the ground-truth data 262, data that can specify the α loop, data that can specify the inverse α loop, or data that can specify the non-loop is associated with the example image set 258A1.

The example image set 258A1 includes a plurality of example images 122A (here, for example, three or more example images 122A) arranged in time series. The example image set 258A1 includes any one of seventh to ninth time-series information.

The seventh time-series information is information in which a plurality of endoscopic images obtained by imaging the inside of the large intestine with the endoscope at a predetermined frame rate (for example, several tens of frames/second) from a time when the insertion part of the endoscope is inserted into the large intestine to a time when the α loop of the insertion part is formed in the endoscopy are arranged in time series as a plurality of example images 122A.

The eighth time-series information is information in which a plurality of endoscopic images obtained by imaging the inside of the large intestine with the endoscope at a predetermined frame rate from a time when the insertion part of the endoscope is inserted into the large intestine to a time when the inverse α loop of the insertion part is formed in the endoscopy are arranged in time series as a plurality of example images 122A.

The ninth time-series information is information in which a plurality of endoscopic images obtained from a time when the insertion part of the endoscope is inserted into the large intestine to a case where the loop of the insertion part is not formed in the endoscopy are arranged in time series as a plurality of example images 122A (for example, the number of frames corresponding to a statistical value such as an average value, a median value, a mode value, a maximum value, or a minimum value of the number of frames obtained until the α loop or the inverse α loop is formed).

Here, as the ground-truth data 262, data that can specify the α loop is associated with the example image set 258A1 including the seventh time-series information. In addition, as the ground-truth data 262, data that can specify the inverse α loop is associated with the example image set 258A1 including the eighth time-series information. Further, as the ground-truth data 262, data that can specify the non-loop is associated with the example image set 258A1 including the ninth time-series information.

In the information processing device 100, the processor 106 executes the machine learning using the dataset group 258. Hereinafter, the details will be described with reference to FIG. 26.

The processor 106 executes processing using a model 264. Examples of the model 264 include the same neural network as the model 142 shown in FIG. 11. The processor 106 acquires the dataset 258A from the dataset group 258. Then, the processor 106 inputs the plurality of example images 122A arranged in time series in the example image set 258A1 included in the dataset 258A acquired from the dataset group 258 to the model 264 in time series. In a case where the plurality of example images 122A arranged in time series are input, the model 264 performs inference and outputs an inference result 266. The processor 106 calculates an error 268 between the inference result 266 and the ground-truth data 262 included in the dataset 258A acquired from the dataset group 258.

The processor 106 calculates a plurality of adjustment values 270 that minimize the error 268. Then, the processor 106 optimizes the model 264 by adjusting a plurality of optimization variables in the model 264 using the plurality of adjustment values 270. Examples of the plurality of optimization variables in the model 264 include a weight and a bias.

The processor 106 repeatedly performs learning processing of inputting the example image set 258A1 to the model 264, calculating the error 268, calculating the plurality of adjustment values 270, and adjusting the plurality of optimization variables in the model 264 using all the datasets 258A included in the dataset group 258. That is, the processor 106 optimizes the model 264 by adjusting the plurality of optimization variables in the model 264 using the plurality of adjustment values 270 calculated to minimize the error 268 for each of all the example image sets 258A1 included in all the datasets 258A. The shape recognition model 260 is generated by optimizing the model 264 in this way. The shape recognition model 260 is transmitted from the information processing device 100 to the medical support device 24 via the external I/Fs 80 and 104 (see FIG. 7), and is received by the medical support device 24. Then, in the medical support device 24, the processor 82 stores the shape recognition model 260 in the storage 86. The shape recognition model 260 stored in the storage 86 is used by the processor 82.

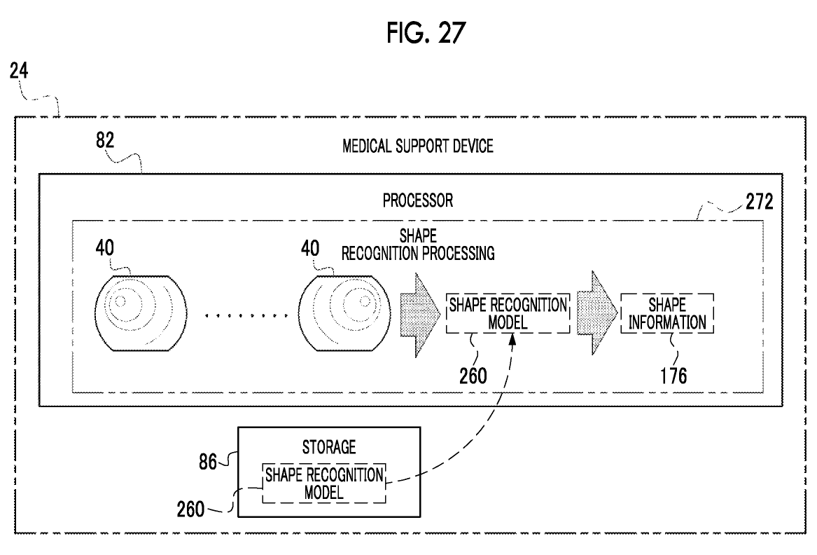

In this case, for example, as shown in FIG. 27, the processor 82 holds the plurality of frames 40 in time series (for example, three or more frames 40). The processor 82 executes shape recognition processing 272 on the plurality of frames 40 in time series. The shape recognition processing 272 is processing of recognizing the shape information 176 from the plurality of frames 40 in time series by using the shape recognition model 260 stored in the storage 86 (that is, the shape recognition model 260 obtained in the manner described using the example shown in FIG. 26) (in other words, processing of specifying whether the shape of the insertion part 48 of the endoscope 16 in the large intestine 28 is the α loop, the inverse α loop, or the non-loop by using the shape recognition model 260).

The processor 82 causes the shape recognition model 260 to generate the shape information 176 by inputting the plurality of frames 40 in time series to the shape recognition model 260 in time series.

In the fourth embodiment, the plurality of frames 40 are examples of “a plurality of endoscopic images” according to the present disclosure. In addition, in the fourth embodiment, the large intestine 28 is an example of a “luminal organ” according to the present disclosure. In addition, in the fourth embodiment, the endoscope 16 is an example of an “endoscope” according to the present disclosure. In addition, in the fourth embodiment, the insertion part 48 is an example of an “insertion part” according to the present disclosure. In addition, in the fourth embodiment, the shape recognition model 260 is an example of a “trained model” according to the present disclosure. In addition, in the fourth embodiment, the shape information 176 is an example of “shape information” according to the present disclosure.

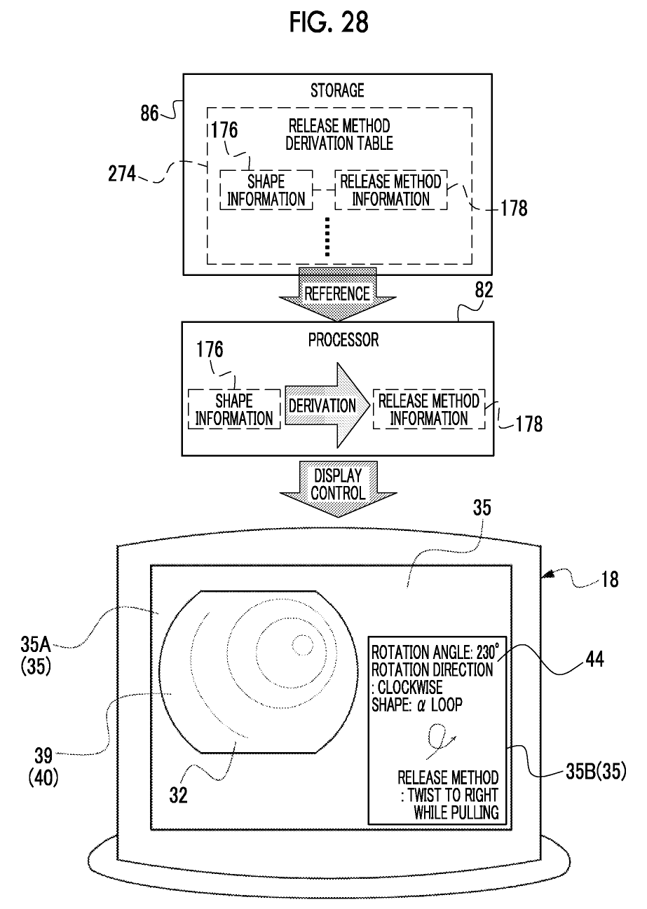

As shown in FIG. 28 as an example, a release method derivation table 274 is stored in the storage 86. The release method derivation table 274 is a table in which the shape information 176 and the release method information 178 in a relationship of being associated with each other are associated with each other. For example, as the release method information 178, information on a method of releasing the α loop is associated with the shape information 176 in a case where the shape information 176 is information that can specify the α loop. In addition, for example, as the release method information 178, information on a method of releasing the inverse α loop is associated with the shape information 176 in a case where the shape information 176 is information that can specify the inverse α loop.

The processor 82 derives the release method information 178 corresponding to the shape information 176 generated in the manner described in the example shown in FIG. 27 from the release method derivation table 274 by referring to the release method derivation table 274. Then, the processor 82 displays the shape information 176, the release method information 178, and the like in the second display region 35B as visible information. In this way, the same effects as those of the above-described embodiment can be obtained.

It should be noted that, in the fourth embodiment, the form example has been described in which the shape information 176 is generated by the shape recognition model 260 and the release method information 178 is derived from the release method derivation table 274, but this is merely an example. For example, both the shape information 176 and the release method information 178 may be generated by a trained model obtained by optimizing the model by performing machine learning on the model for both the shape (for example, the α loop, the inverse α loop, and the non-loop) and the release method (for example, the method of releasing the α loop and the method of releasing the inverse α loop).

[Fifth Embodiment] In the fourth embodiment, the form example has been described in which the shape information 176 is derived by the AI method using the plurality of frames 40 in time series, but this is merely an example. In the fifth embodiment, a form example will be described in which the shape information 176 is derived by the AI method using the plurality of pieces of lumen position information 152A1a in time series. In the fifth embodiment, constituents described in the first to fourth embodiment will be designated by the same reference numerals and will not be described, and different parts from the first to fourth embodiment will be described.

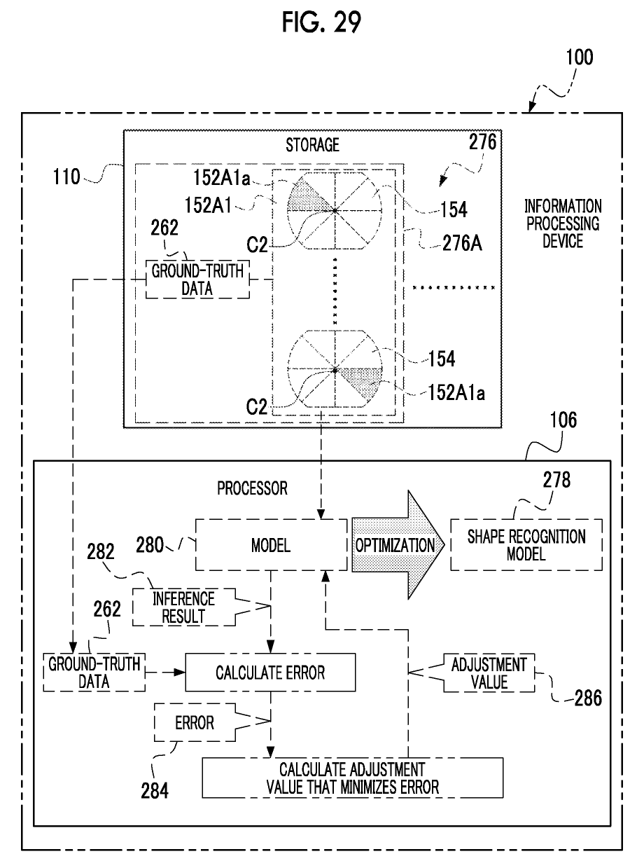

FIG. 29 is a conceptual diagram showing an example of an aspect in which the shape recognition model 278 is generated by performing machine learning using a dataset group 276 by the processor 106.

As shown in FIG. 29, the dataset group 276 is a set of a plurality of datasets 276A. The plurality of datasets 276A have different contents. The dataset 276A is training data in which the lumen position information set 152A1 and the ground-truth data 262 described in the fourth embodiment are associated with each other. The dataset 276A is generated by associating the ground-truth data 262 with the lumen position information set 152A1 in the same manner as the training data 128 shown in FIG. 8 is generated by the training data generation unit 106A.

As described in the first embodiment, the lumen position information set 152A1 includes any one of first to third time-series information. As the ground-truth data 262, data that can specify the α loop is associated with the lumen position information set 152A1 including the first time-series information. In addition, as the ground-truth data 262, data that can specify the inverse α loop is associated with the lumen position information set 152A1 including the second time-series information. Further, as the ground-truth data 262, data that can specify the non-loop is associated with the lumen position information set 152A1 including the third time-series information.

In the information processing device 100, the processor 106 executes the machine learning using the dataset group 276. Hereinafter, the details will be described with reference to FIG. 29.

The processor 106 executes processing using a model 280. Examples of the model 280 include the same neural network as the model 142 shown in FIG. 11. The processor 106 acquires the dataset 276A from the dataset group 276. Then, the processor 106 inputs the plurality of pieces of lumen position information 152A1a arranged in time series in the lumen position information set 152A1 included in the dataset 276A acquired from the dataset group 276 to the model 280 in time series. In a case where the plurality of pieces of lumen position information 152A1a arranged in time series are input, the model 280 performs inference and outputs an inference result 282. The processor 106 calculates an error 284 between the inference result 282 and the ground-truth data 262 included in the dataset 276A acquired from the dataset group 276.

The processor 106 calculates a plurality of adjustment values 286 that minimize the error 284. Then, the processor 106 optimizes the model 280 by adjusting a plurality of optimization variables in the model 280 using the plurality of adjustment values 286. Examples of the plurality of optimization variables in the model 280 include a weight and a bias.

The processor 106 repeatedly performs learning processing of inputting the dataset 276A to the model 280, calculating the error 284, calculating the plurality of adjustment values 286, and adjusting the plurality of optimization variables in the model 280 using all the datasets 276A included in the dataset group 276. That is, the processor 106 optimizes the model 280 by adjusting the plurality of optimization variables in the model 280 using the plurality of adjustment values 286 calculated to minimize the error 284 for each of all the lumen position information sets 152A1 included in all the datasets 276A. The shape recognition model 278 is generated by optimizing the model 280 in this way. The shape recognition model 278 is transmitted from the information processing device 100 to the medical support device 24 via the external I/Fs 80 and 104 (see FIG. 7), and is received by the medical support device 24. Then, in the medical support device 24, the processor 82 stores the shape recognition model 278 in the storage 86. The shape recognition model 278 stored in the storage 86 is used by the processor 82.

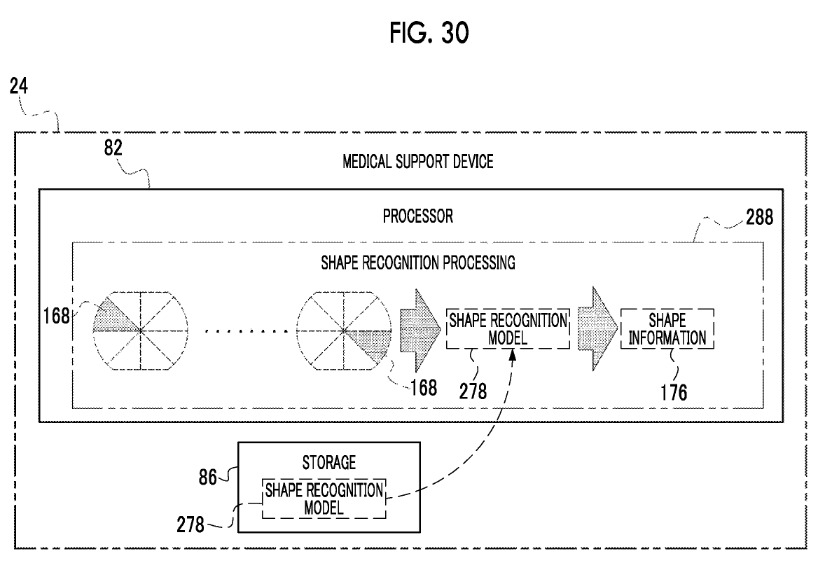

In this case, for example, as shown in FIG. 30, the processor 82 holds the plurality of pieces of lumen position information 168 in time series in the same manner as in the example shown in FIG. 15 of the first embodiment. The processor 82 executes shape recognition processing 288 on the plurality of pieces of lumen position information 168 in time series (here, for example, three or more pieces of lumen position information 168). The shape recognition processing 288 is processing of recognizing the shape information 176 from the plurality of pieces of lumen position information 168 in time series by using the shape recognition model 278 stored in the storage 86 (that is, the shape recognition model 278 obtained in the manner described using the example shown in FIG. 29) (in other words, processing of specifying whether the shape of the insertion part 48 of the endoscope 16 in the large intestine 28 is the α loop, the inverse α loop, or the non-loop by using the shape recognition model 278).

The processor 82 causes the shape recognition model 278 to generate the shape information 176 by inputting the plurality of pieces of lumen position information 168 in time series to the shape recognition model 278 in time series. Then, in the fifth embodiment, the same processing as in the example shown in FIG. 28 described in the fourth embodiment is executed by the processor 82. In this way, the same effects as those of the above-described embodiment can be obtained.

In the fifth embodiment, the plurality of frames 40 are examples of “a plurality of endoscopic images” according to the present disclosure. In addition, in the fifth embodiment, the plurality of pieces of lumen position information 168 are examples of “the accumulated result in which changes in feature information included in each of a plurality of endoscopic images obtained by imaging the inside of the luminal organ with the endoscope whose insertion part is inserted into the luminal organ between the plurality of endoscopic images are accumulated”. In addition, in the fifth embodiment, the large intestine 28 is an example of a “luminal organ” according to the present disclosure. In addition, in the fifth embodiment, the endoscope 16 is an example of an “endoscope” according to the present disclosure. In addition, in the fifth embodiment, the insertion part 48 is an example of an “insertion part” according to the present disclosure. In addition, in the fifth embodiment, the shape recognition model 278 is an example of a “trained model” according to the present disclosure. In addition, in the fifth embodiment, the shape information 176 is an example of “shape information” according to the present disclosure.

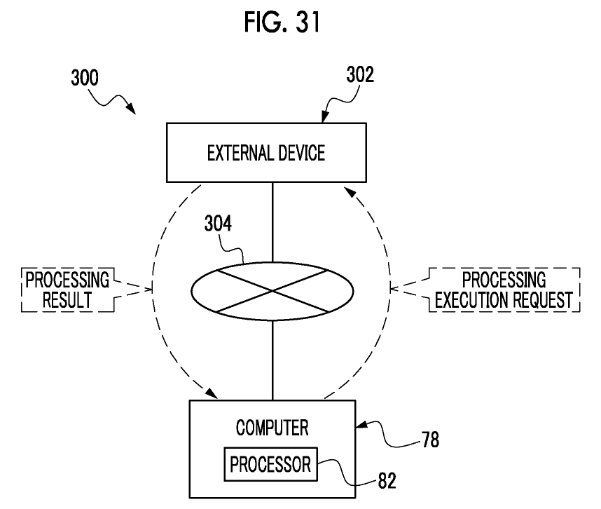

In each of the embodiments described above, a form example has been described in which the medical support process is performed by the computer 78, but the present disclosure is not limited to this. At least some of processing included in the medical support process may be performed by a device provided outside the computer 78. Hereinafter, an example of this case will be described with reference to FIG. 31.

FIG. 31 is a conceptual diagram showing an example of a configuration of an endoscope system 300. The endoscope system 300 is an example of an "endoscope system" according to the present disclosure. The endoscope system 300 is different from the endoscope system 10 according to the above-described embodiment in that an external device 302 is provided.

The external device 302 is connected communicably to the computer 78 via a network 304 (for example, a WAN and/or a LAN).

Examples of the external device 302 include at least one server that directly or indirectly transmits and receives data to and from the computer 78 via the network 304. The external device 302 receives a processing execution instruction issued from the processor 82 of the computer 78 via the network 304. Then, the external device 302 executes processing corresponding to the received processing execution instruction, and transmits a processing result to the computer 78 via the network 304. In the computer 78, the processor 82 receives the processing result transmitted from the external device 302 via the network 304, and executes processing using the received processing result.

Examples of the processing execution instruction include an instruction for the external device 302 to execute at least a part of the medical support processing.

A first example of the at least a part of the medical support processing (that is, processing to be executed by the external device 302) is the lumen recognition processing 166. In this case, the external device 302 executes the lumen recognition processing 166 in response to the processing execution instruction issued from the processor 82 via the network 304, and transmits the lumen position information 168 to the computer 78 via the network 304. In the computer 78, the processor 82 receives the lumen position information 168 and executes processing using the received lumen position information 168.

A second example of at least a part of the medical support processing (that is, processing executed on the external device 302) is the rotation recognition processing 172, 238, or 256. In this case, the external device 302 executes the rotation recognition processing 172, 238, or 256 in response to the processing execution instruction given from the processor 82 via the network 304, and transmits the rotation information 174 to the computer 78 via the network 304. In the computer 78, the processor 82 receives the rotation information 174 and executes processing using the received rotation information 174.

A third example of at least a part of the medical support processing (that is, processing executed on the external device 302) is the shape recognition processing 272 or 288. In this case, the external device 302 executes the shape recognition processing 272 or 288 in response to the processing execution instruction given from the processor 82 via the network 304, and transmits the shape information 176 to the computer 78 via the network 304. In the computer 78, the processor 82 receives the shape information 176 and executes processing using the received shape information 176.

A fourth example of the at least a part of the medical support processing (that is, processing to be executed by the external device 302) is the gravity direction recognition processing 230. In this case, the external device 302 executes the gravity direction recognition processing 230 in response to the processing execution instruction given from the processor 82 via the network 304, and transmits the gravity direction information 232 to the computer 78 via the network 304. In the computer 78, the processor 82 receives the gravity direction information 232 and executes processing using the received gravity direction information 232.

A fifth example of at least a part of the medical support processing (that is, processing executed on the external device 302) is processing by the control unit 82B (for example, the processing described in each of the above-described embodiments). In this case, the external device 302 executes the processing by the control unit 82B in response to the processing execution instruction given from the processor 82 via the network 304, and transmits a processing result (for example, visible information to be displayed on the screen 35) to the computer 78 via the network 304. In the computer 78, the processor 82 receives the processing result and executes the same processing as the processing in the above-described embodiment using the received processing result.

The external device 302 may be implemented by cloud computing. The cloud computing is merely an example, and the external device 302 may be implemented by network computing, such as fog computing, edge computing, or grid computing.

In each of the above-described embodiments, each processing is executed by any computer. Moreover, any computer may execute these processes by a processor as hardware, a program as software, or a combination thereof. In that case, the processor is configured to execute various types of processes in the above-described embodiments in cooperation with the program, and can function as each unit or each means in the present embodiment. Further, the execution order of the processing by the processor is not limited to the above-described order and may be changed as appropriate. Any computer may be a general-purpose computer, a computer for a specific use, a workstation, or another system capable of executing each processing.

The processor may be configured by one or a plurality of pieces of hardware, and the type of hardware is not limited. For example, the processor may be configured by a CPU, an MPU, a programmable logic device such as an FPGA, a dedicated circuit for executing specific processing such as an ASIC, a GPU, an NPU, or hardware. Types of hardware may be a combination of different types of hardware. In a case where a plurality of hardware are configured to execute one or a plurality of processes of a certain processor, the plurality of hardware may be present in devices physically separated from each other, or may be present in the same device. In any embodiment, the order of each processing via the processor is not limited to the above order and may be appropriately changed. The hardware is configured using an electrical circuit (circuitry) in which circuit elements, such as semiconductor elements, are combined, or the like.

Further, the program may be software such as firmware or microcode. For example, the program may be a program module group, and each function thereof may be implemented by a processor configured to execute each function. The program may be a program code or a plurality of code segments stored in one or a plurality of non-transitory computer-readable media (for example, a storage medium and/or other storage). The program may be divided and stored in a plurality of non-transitory computer-readable media present in apparatuses physically separated from each other. The program code or the code segment may represent any combination of a procedure, a function, a subprogram, a routine, a subroutine, a module, a software package, a class, an instruction, a data structure, or a program statement. The program code or code segment may be connected to another code segment or a hardware circuit by transmitting and receiving information, data, an argument, a parameter, or a content of a memory.

In addition, in each of the above-described embodiments, the aspect example (that is, the aspect example of being installed) has been described in which the medical support program 90 or 200 (hereinafter, referred to as a “medical support program” without reference numerals) is stored in advance in the storage 86, but the present disclosure is not limited thereto. The medical support program may be provided in a form of being stored in a storage medium such as a CD-ROM, a DVD-ROM, and a USB memory. In addition, the medical support program may be downloaded from an external device via a network.

The technology of the present disclosure extends to any program products. The program product includes products of every aspect for providing the program. For example, the program product includes a program provided through a network such as the Internet, and non-transitory computer-readable storing media such as a CD-ROM, a DVD, and a USB memory in which the program is stored.

The above-described medical support processing is merely an example. Accordingly, it is possible to delete an unnecessary step, add a new step, or change a processing order without departing from the gist of the present disclosure.

The above-described contents and the above-shown contents are the detailed description of the parts according to the present disclosure, and are merely examples of the present disclosure. For example, description related to the above configurations, functions, actions, and effects is description related to an example of configurations, functions, actions, and effects of the parts relating to the present disclosure. Therefore, it goes without saying that unnecessary parts may be deleted, new elements may be added, or replacements may be made with respect to the above-described contents and the above-shown contents within a range that does not deviate from the gist of the present disclosure. In addition, in order to avoid complications and facilitate understanding of the parts according to the present disclosure, the description of common technical knowledge or the like, which does not particularly require the description for enabling the implementation of the present disclosure, is omitted in the above-described contents and the above-shown contents.

All documents, patent applications, and technical standards mentioned in the present specification are incorporated herein by reference to the same extent as in a case in which each document, each patent application, and each technical standard are specifically and individually described by being incorporated by reference.

What is claimed is:

1. A medical support device comprising:
a processor,
wherein the processor is configured to:
input a plurality of endoscopic images obtained by imaging an inside of a luminal organ, by using an endoscope which is inserted into the luminal organ, to a trained model to cause the trained model to generate rotation information of an insertion part of the endoscope around a major axis; and
generate shape information representing a shape of the insertion part based on the rotation information.

2. The medical support device according to claim 1,
wherein the rotation information is obtained based on an accumulated result in which changes in feature information obtained from the endoscopic image between the plurality of endoscopic images are accumulated.

3. The medical support device according to claim 2,
wherein the feature information includes lumen position information indicating a position of a lumen included in the luminal organ in the endoscopic image, and
the rotation information is obtained based on an accumulated result in which changes in the lumen position information between the plurality of endoscopic images are accumulated.

4. The medical support device according to claim 2,
wherein the feature information includes gravity direction information for specifying a gravity direction, and
the rotation information is obtained based on an accumulated result in which changes in the gravity direction information between the plurality of endoscopic images are accumulated.

5. The medical support device according to claim 1,
wherein the rotation information includes information on a rotation angle and a rotation direction of a second position, which are relative with respect to a first position of the insertion part, or information on a rotation angle and a rotation direction of the first position and the second position, which are absolute with respect to a reference angle.

6. The medical support device according to claim 1,
wherein the shape information includes information indicating that the insertion part forms a loop in a case where a distal end position of the insertion part is rotated by 180 degrees or more with respect to a base end position of the insertion part.

7. The medical support device according to claim 6,
wherein the rotation information includes rotation direction information for specifying a rotation direction around the major axis, and
the shape information includes loop classification information that classifies a shape of the loop based on the rotation direction information.

8. The medical support device according to claim 7,
wherein the loop classification information includes information that classifies the loop into an α loop or an inverse α loop based on the rotation direction information.

9. The medical support device according to claim 6,
wherein the processor is configured to output information on a method of releasing the loop based on the rotation information and/or the shape information.

10. The medical support device according to claim 1,
wherein the processor is configured to input the plurality of endoscopic images and a plurality of images including a hand-held part of an operator in the insertion part to the trained model to cause the trained model to generate information based on information on rotation of the hand-held part around the major axis as the rotation information.

11. The medical support device according to claim 1,
wherein the luminal organ is a large intestine.

12. A medical support device comprising:
a processor,
wherein the processor is configured to input a plurality of endoscopic images obtained by imaging an inside of a luminal organ, by using an endoscope which is inserted into the luminal organ, to a trained model to cause the trained model to generate shape information representing a shape of an insertion part of the endoscope.

13. A medical support device comprising:
a processor,
wherein the processor is configured to input an accumulated result in which changes in feature information between a plurality of endoscopic images are accumulated, the feature information being included in each of the plurality of endoscopic images obtained by imaging an inside of a luminal organ, by using an endoscope which is inserted into the luminal organ, to a trained model to cause the trained model to generate shape information representing a shape of an insertion part of the endoscope.

14. An endoscope system comprising:
the medical support device according to claim 1; and
an output device that outputs the shape information generated by the medical support device and/or information based on the shape information generated by the medical support device.

15. A medical support method comprising:
inputting a plurality of endoscopic images obtained by imaging an inside of a luminal organ, by using an endoscope which is inserted into the luminal organ, to a trained model to cause the trained model to generate rotation information of an insertion part of the endoscope around a major axis; and
generating shape information representing a shape of the insertion part based on the rotation information.

16. A non-transitory computer-readable storage medium storing a program executable by a computer to execute a process comprising:
inputting a plurality of endoscopic images obtained by imaging an inside of a luminal organ, by using an endoscope which is inserted into the luminal organ, to a trained model to cause the trained model to generate rotation information of an insertion part of the endoscope around a major axis; and
generating shape information representing a shape of the insertion part based on the rotation information.
